(19) United States
(12) Patent Application Publication
(10) Pub. No.: US 2009/0318752 A1
(43) Pub. Date: Dec. 24, 2009

(54) IMPLANTS FOR THE TREATMENT OF PELVIC FLOOR DISORDERS

(75) Inventors: Douglas G. Evans, Snellville, GA (US); Walter Freitag, Atlanta, GA (US)

Correspondence Address: Rutan & Tucker, LLP., 611 ANTON BLVD, SUITE 1400, COSTA MESA, CA, 92626, US

(73) Assignee: C. R. Bard, Inc., Murray Hill, NJ (US)

(21) Appl. No.: 12/282,641

(22) PCT Filed: Mar. 15, 2007
(86) PCT No.: PCT/US07/06461

§ 371 Date: Dec. 4, 2008

Related U.S. Application Data
(60) Provisional application No. 60/743,496, filed on Mar. 15, 2006.

(30) Foreign Application Priority Data
Aug. 3, 2006 (US) PCT/US06/30359
Aug. 3, 2006 (US) PCT/US06/30370
Aug. 3, 2006 (US) PCT/US06/30581
Dec. 28, 2006 (US) PCT/US06/62639

(51) Int. Cl. A61F 2/02

(52) U.S. Cl. 600/37

(57) ABSTRACT

An implant for repairing pelvic floor disorders, such as urinary incontinence or prolapse, by lifting the prolapsed tissue or organ into a more anatomically correct position is provided. The prosthesis is shaped with a central support section and one or more lateral anchoring sections or arms extending outwardly from the central section. The support section lifts the prolapsed organ to a more anatomically correct position, whereas the anchoring secton(s) are positioned through soft tissue away from the organ being supported to hold the implant in place by allowing tissue ingrowth into the anchoring sections. The implant may be formed of both synthetic and more natural materials.

### CROSS-REFERENCE TO RELATED APPLICATIONS

This application claims the benefit of U.S. Provisional Patent Application Ser. No. 60/743,496, filed Mar. 15, 2006; PCT Application No. PCT/US2006/030581, filed Aug. 3, 2006; PCT Application No. PCT/US2006/030369, filed Aug. 3, 2006; PCT Application No. PCT/US2006/030370, filed Aug. 3, 2006; and PCT Application No. PCT/US2006/062639, filed Dec. 28, 2006; the disclosures of which are all incorporated herein by reference in their entirety.

### FIELD OF INVENTION

The present invention relates to an implantable prosthesis and, more particularly, to an implant for the treatment of pelvic floor disorders.

### DISCUSSION OF RELATED ART

Pelvic implants are used to treat various disorders. For example, pelvic implants are used to perform prolapse repair, such as cystocele repair and/or rectocele repair, and to treat urinary incontinence. Implants may be shaped to correct the disorder. However, some shaped implants do not lend themselves to ease of implantation and often, as a result of their shape, will not lie in a suitable position and/or orientation to correct the defect. Implants are often formed of a knitted mesh that, once implanted, enables the ingrowth of soft tissue to fix the implant in place.

Prior implants are formed of synthetic materials or from natural, biologically derived materials. Synthetic materials are desirable in some situations because they do not degrade within the body, allowing the resulting implant to provide a permanent support after implantation. Typical synthetic materials include polypropylene knitted mesh. Some synthetic implants can elicit a foreign body reaction, harbor infections, or cause erosion through the host tissue. Erosion occurs when the modulus of elasticity of the synthetic material is different than that of the surrounding soft tissue in the body, leading to abrasions between the synthetic implant and the surrounding host tissue and causing the synthetic material to wear through the host tissue.

Implants formed of natural materials tend to have greater biocompatibility and therefore have a less chance of harboring infections, eliciting a foreign body reaction or eroding through tissue. A trade-off with using a natural material for the implant is that they are often not permanent implants, meaning that the body eventually degrades and breaks down the implant via normal biological processes, such as collagenase, resulting in the recurrence of the condition being treated. Advances in bioengineering has allowed for improved natural materials to be developed to extend the time required for breakdown (such as by cross-linking of biologic tissue).

Aspects of the invention are directed to improved implants.

### SUMMARY OF THE INVENTION

In one embodiment, an implant is provided. The implant includes a body portion configured to support an organ. The body portion defines a central axis and a first end area at one end of the central axis and a second end area at an opposite end of the central axis. The body portion has lateral side edges. A projection extends from the first end area of the body portion in a direction substantially parallel to the central axis. The projection has lateral side edges and an end opposite the first end area. At least three anchoring arms extend from the body portion, with at least one first arm extending outward from the first end area and at least one second arm extending outward from the second end area. The lateral side edges of the body portion diverge in a direction from the second end area toward the first end area and the lateral side edges of the projection diverge in a direction from the end of the projection toward the first end area, whereby the first end area has a width that is wider than: a width of the second end area, and a width of the end of the projection.

In yet another embodiment, an implant is provided. The implant includes a body portion configured to support an organ. The body portion defines a central axis and a first end area at one end of the central axis and a second end area at an opposite end of the central axis. The body portion has lateral side edges. A projection extends from the first end area of the body portion in a direction substantially parallel to the central axis. The projection has lateral side edges and an end opposite the first end area. A first pair of arms extends from the body portion on opposite sides of the first end area and a second pair of arms extending from the body portion on opposite sides of the second end area. Each arm of the first pair of arms extends substantially perpendicular to the central axis and each arm of the second pair of arms extends at an angle relative to the central axis.

In still another embodiment, an implant is provided. The implant includes a body portion configured to support an organ. The body portion defines a central axis and a first end area at one end of the central axis and a second end area at an opposite end of the central axis. The body portion has lateral side edges. A projection extends from the first end area of the body portion in a direction substantially parallel to the central axis. The projection has lateral side edges and an end opposite the first end area. A first pair of arms extends from the body portion on opposite sides of the first end area and a second pair of arms extends from the body portion on opposite sides of the second end area. Each arm has a folded end portion.

In yet another embodiment, an implant is provided. The implant includes a support layer. The support layer has a body portion configured to support an organ. The body portion defines a central axis and a first end area at one end of the central axis and a second end area at an opposite end of the central axis. The body portion has lateral side edges. A projection extends from the first end area of the body portion in a direction substantially parallel to the central axis. The projection has lateral side edges and an end opposite the first end area. At least three anchoring arms extend from the body portion, with at least one first arm extending outward from the first end area and at least one second arm extending outward from the second end area. A layer of natural material is disposed on a first surface of at least a portion of the support layer. The first layer of natural material is spaced from the lateral side edges of the both the body portion and the projection.

In still another embodiment, an implant is provided. The implant includes a support layer. The support layer has a body portion configured to support an organ. The body portion defines a central axis and a first end area at one end of the central axis and a second end area at an opposite end of the central axis. The body portion has lateral side edges. A projection extends from the first end area of the body portion in a direction substantially parallel to the central axis. The projection has lateral side edges and an end opposite the first end area. At least three anchoring arms extend from the body portion, with at least one first arm extending outward from the first end area and at least one second arm extending outward from the second end area. A layer of natural material is disposed on at least a portion of the support layer. The layer of natural material is attached to the support layer with criss-crossing stitches resulting in a checkerboard pattern.

Various embodiments of the present invention provide certain advantages and overcome certain drawbacks of prior implants. Embodiments of the invention may not share the same advantages, and those that do may not share them under all circumstances.

Further features and advantages of the present invention, as well as the structure of various embodiments, are described in detail below with reference to the accompanying drawings.

### BRIEF DESCRIPTION OF THE DRAWINGS

The accompanying drawings are not intended to be drawn to scale. In the drawings, similar features are represented by like reference numerals. For purposes of clarity, not every component is labeled in every drawing. Various embodiments of the invention will now be described, by way of example, with reference to the accompanying drawings, in which:

FIG. 1 is a top view of an embodiment of an anterior implant;

FIG. 1A is a perspective view of the implant of FIG. 1;

FIG. 2 is a top view of an embodiment of a posterior implant;

FIG. 2A is a perspective view of the implant of FIG. 2;

FIG. 3 is a top view of another embodiment of a posterior implant;

FIG. 3A is a perspective view of the implant of FIG. 3;

FIGS. 4A and 4B are plan views of alternative embodiments of a portion of an implant, showing alternative embodiments of an apical flap;

FIG. 5 is a view of a portion of the implant shown in FIG. 3;

FIG. 6 is a plan view of alternative embodiment of a portion of an implant, showing a reduced arm width;

FIG. 7 is a perspective view of alternative embodiment of a portion of an implant, showing a folded arm;

FIG. 8 is a plan view of alternative embodiment of a portion of an implant, showing visual indicators;

FIGS. 9A-9C are plan views of alternative embodiments of a portion of an implant, showing an attached natural material layer;

FIGS. 10A-10K are schematic representations of alternative embodiments showing characteristics of holes and hole patterns formed through the natural material layer;

FIG. 11 is a schematic representation of one embodiment of a hole pattern through the natural material layer;

FIGS. 12A-12D are schematic representations of alternative stitch patterns for attaching the natural material layer;

FIGS. 13-13A are schematic representations of an alternative attachment arrangement for attaching the natural material layer;

FIG. 14 is a schematic representation of another embodiment of a stitch pattern for attaching the natural material layer;

FIG. 15 is a schematic representation of an embodiment of a stitch pattern for attaching the natural material layer to an anterior implant;

FIG. 16 is a schematic representation of an embodiment of a stitch pattern for attaching the natural material layer to a posterior implant;

FIG. 17 is a top view of an embodiment of an anterior introducer suitable for use in implanting an anterior implant;

FIG. 18 is a side view of the anterior introducer of FIG. 17;

FIG. 19 is a cross-sectional view of a needle of the anterior introducer of FIG. 18 taken along lines 19-19;

FIG. 20 is a top view of an embodiment of a posterior introducer suitable for use in implanting a posterior implant;

FIG. 21 is a side view of the posterior introducer of FIG. 20;

FIG. 22 is a detail view of a portion of the anterior introducer needle of FIGS. 20 and 21;

FIGS. 23A-23F represent an illustrative procedure for implanting an anterior implant;

FIGS. 24A-24L represent an illustrative procedure for implanting a posterior implant; and

FIG. 25 is a perspective view of an implant;

FIG. 26 is a cross-sectional view of the implant of FIG. 18 taken along lines 25-25;

FIG. 27 is a perspective view an embodiment of an introducer system; and

FIG. 28 illustrates passage of a snare through the introducer, shown in FIG. 27.

### DESCRIPTION OF ILLUSTRATIVE EMBODIMENTS

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

According to one aspect of the invention, an implantable prosthesis for an anatomical defect and methods for the treatment of such defects is provided. The implant may be configured for use as a prosthesis to repair pelvic floor disorders, such as urinary incontinence or prolapse, by lifting the prolapsed tissue or organ into a more anatomically correct position. The types of prolapse contemplated include rectoceles, cystoceles (whether central or lateral), enteroceles, urethroceles, hysteroceles, vaginal vault prolapse, uterine prolapse, and/or any other prolapse or combination of such disorders. Also, the implant may be configured to treat pelvic disorders of both males and females.

In one embodiment, the prosthesis is formed with a central support section and one or more lateral anchoring sections or arms extending outwardly from the central section. The support section is constructed in a manner to provide support to the tissue defect, such as by lifting the prolapsed organ to a more anatomically correct position, and the anchoring section(s) are positioned through soft tissue away from the organ being supported and are used to hold the implant in place by allowing tissue ingrowth into the anchoring sections. Tissue ingrowth through the arm sections anchors the implant, providing a “hammock” like support to the central area.

It should be appreciated that the present invention is not limited to prolapse repair of pelvic organs. Rather, the implant may be used to aid in the repair of other tissue or muscle defects or incisions, including but not limited to hernias or torn ligaments, or may be used as a graft, an anastomosis wrap or other gastric band, all whether located in the pelvic area, abdominal area or other areas, as the present invention is not limited in this respect.

In one embodiment, the implant is formed of a synthetic material that may be formed from a sheet of knitted polypropylene monofilament mesh fabric, such as BARD MESH available from C. R. Bard, Inc. When implanted, the polypropylene mesh promotes tissue or muscle ingrowth into and around the mesh structure. As will be described below, other suitable materials may be employed, as the present invention is not limited in this respect.

According to another aspect of the invention, a hybrid implant formed of both synthetic and more natural materials is provided. In this manner, the synthetic material aids in providing the permanence of implant as the underlying support, while the more natural material acts as a buffer between the synthetic material and host tissue of the patient.

By having the synthetic mesh layer as the supportive portion under the tissue, the strength of the more natural material is not as important for maintaining support of the target organ or surrounding soft tissue, as this function is primarily provided by the synthetic mesh. In this regard, the support layer extends over the full width of the implant, providing a permanent support underneath the host tissue. Prior attempts at making hybrid implants have focused on having synthetic mesh arms, while the central organ support section is formed of natural material only. A disadvantage with this design is that the natural material will degrade leaving behind only the anchoring arms, reducing or entirely eliminating the supportive function of the implant to support the organ and resulting in a high recurrence of the herniation of the organ.

In one embodiment, the implant is formed of a synthetic material with the more natural material formed as a separate element attached to the support section of the implant, such that the prolapsed organ or tissue can rest against the more natural material. The materials may be joined together by a variety of techniques, such as mechanical, thermal, chemical, or other suitable attachment techniques, as the present invention is not limited in this respect.

In one embodiment, the implant includes multiple layers of different materials; for example, a support layer (such as a support formed of a synthetic mesh material) sandwiched between two layers of more natural material. It should be appreciated that the present invention is not limited in this respect, as the implant need only be provided with a more natural material on one side of the synthetic material.

The combination of the synthetic and the more natural materials creates a hybrid implant having the benefits of a buffer on one or both sides of the support layer, minimizing the risk of erosion into the organ being supported. The support material provides a permanent support for repairing the defect, even after the more natural material buffers are degraded by the body.

In one embodiment, the synthetic material extends over the full width and length of the implant, essentially creating the outer profile or shape of the implant. The layer of the more natural material (on one or both sides) cover only the portion of the graft where the organ is to be supported, such as the in the center region of the implant.

It should be appreciated that the more natural material layer may be formed of a purely natural material or may be an engineered material designed to mimic certain attributes of a purely natural material. Accordingly, the term natural material used herein is intended to encompass such materials.

The prosthesis includes various features, each of which will be described in greater detail below, that may be employed singularly or in any suitable combination, as the present invention is not limited in this respect.

Illustrative embodiments of implants suitable for use in treating prolapse will now be disclosed. The implants each have a body portion and arms that extend from the body portion. The body portion can be positioned adjacent an organ to be supported, such as the bladder, rectum, uterus, or urethra. Generally speaking, the implants are formed from a flexible material. As discussed, the implant material can be formed of a synthetic material, a natural material, or a combination of both synthetic and natural materials. Irrespective of the material used to construct the implants, the implants are highly flexible yet have the strength needed for tension-free fixation of the implant.

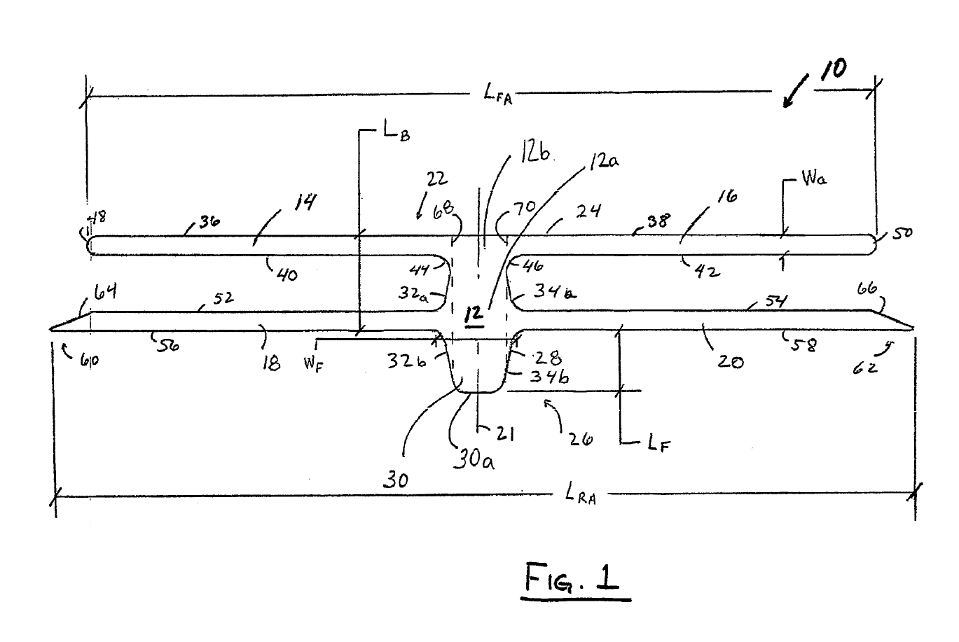

FIGS. 1 and 1A illustrate an embodiment of an anterior implant 10, which may be used to treat cystocele. As indicated in FIG. 1, the implant 10 generally comprises a body portion 12, two front arms 14 and 16, and two rear arms 18 and 20. The body portion 12 defines a central axis 21 about which the implant 10 is substantially symmetric and generally comprises to a front side 22 defined by front edge 24 and a rear side 26 defined by a rear edge 28. In the illustrated embodiment, the front edge 24 is substantially straight while the rear edge 28 forms a rear projection 30 that extends rearwardly from the rear arms 18, 20. The body portion 12 includes a first end area 12A and a second end area 12B and the projection 30 has an end 30A. A pair of arms extends from the first end area and a pair of arms extends from the second end area. The body portion 12 further includes lateral edges 32A and 34A and lateral edges 32B and 34B formed on projection 30. As is apparent from FIG. 1, the lateral edges 32A, 34A extend in a divergent manner from the first end area 12B to the second end area 12B of the body portion 12 (i.e., from the front arms 14, 16 to the rear arms 18, 20) at an angle relative to the central axis 21 such that the body portion 12 widens from the second end area 12B to the first end area 12A, whereas lateral edges 32B, 34B of projection 30 extend in a divergent manner from the end 30A to the first end area 12A at an angle relative to the central axis 21 such that the rear portion 30 widens from the first end 30A to the first end area 12A. With the above-described configuration, the body portion 12 is shaped and configured to support the bladder with the front edge 24 positioned adjacent the bladder neck and the rear flap 30 positioned relatively deep within the pelvis. In such a position, the rounded rear edges of the flap 30 are adapted to accommodate the contours of the pelvis interior. The rear projection 30, which may also be referred to as an apical flap 30, is constructed to support the upper portion of the vagina. Portions of the apical flap 30 can be trimmed or the entire apical flap can be removed by the surgeon, as desired, simply by cutting the mesh. In this regard, the surgeon can trim the implant so as to size it more appropriately to the patient's vaginal length. By way of example, the body portion 12 has a length L_{B} (along the central axis 21) of approximately 60 mm and the rear flap 30 has a length L_{F} F (along the central axis) of approximately 40 mm and the width W_{F} of the flap is approximately 60 mm-70 mm where it joins the body at the first end area 12A.

The front arms 14, 16 extend laterally from the front side 22 of the body portion 12 substantially perpendicular to the central axis 21. In the illustrated embodiment, the front arms 14, 16 are substantially parallel to each other and comprise front edges 36 and 38 that 25 are contiguous with the front edge 24 of the body portion 12. In addition, the front arms 14, 16 comprise rear edges 40 and 42 that are, in some embodiments, substantially parallel to the front edges 36, 38 and to each other. The rear edges 40, 42 join the body portion 12 at rounded inner corners 44 and 46. As is further depicted in FIG. 1, the front arms 14, 16 terminate in rounded ends 48 and 50. By way of example, the front arms 14, 16 span a length L_{FA} of approximately 480 mm.

With continued reference to FIG. 1, the rear arms 18, 20 also extend laterally from the body portion 12 substantially perpendicular to the central axis 21. In the embodiment of FIG. 1, the rear arms 18, 20 are substantially parallel to the front arms 14, 16 and to each other. The rear arms 18, 20 comprise front edges 52 and 54 and rear edges 56 and 58. In some embodiments, the front edges 52, 54 and the rear edges 56, 58 are substantially parallel.

Like the front arms 14, 16, the rear arms 18, 20 terminate in ends 60 and 62. Unlike the ends 48 and 50, however, the ends 60 and 62 of the rear arms 18, 20 are pointed due to the provision of angled edges 64 and 66 that extend from the front edges 52, 54 of the arms 18, 20 to the rear edges 56, 58 of the arms. In the illustrated embodiment, the angled edges 64, 66 are angled outwardly from the front of the arms 18, 20 to the rear. As described below, the pointed ends 60, 62 both facilitate threading of the rear arms 18, 20 through an introducer needle as well as aiding the surgeon in distinguishing the rear arms from the front arms 14, 16. By way of example, the rear arms span a length L_{RA} of approximately 560 mm so as to be slightly longer than the front arms 14, 16. Also, by way of example, each arm has a width WA of approximately 12 mm. In one embodiment, the width of the central section 12 where it joins arms 14, 16 is approximately 450 mm.

In some embodiments all or at least a portion of the body portion 12 is formed of a relatively soft material as compared to the relatively coarse material of the arms 14, 16, 18, and 20 such that the portion of the implant 10 that supports the bladder is relatively soft and compliant while the arms are less compliant to ensure secure fixation and avoid implant migration and/or elongation. In the embodiment of FIG. 1, a central portion defined by the area bounded by dashed lines 68 and 70 (which extend from the front side 22 to the rear side 26 of the body portion 12) having a distance between them of approximately 50 mm comprises the relatively soft material. In addition, as will be discussed below, the body portion 12, on one or both sides, can be provided with a natural layer of material. In the embodiment of FIG. 1, the natural layer covers the area bounded by the hidden lines 72 and 74 (which extend from the front side 22 to the rear side 26 of the body portion 12).

FIG. 1A shows the anterior implant 10 in a perspective view in free space. The flexibility of the implant 10 is apparent from the illustrated orientation of the implant.

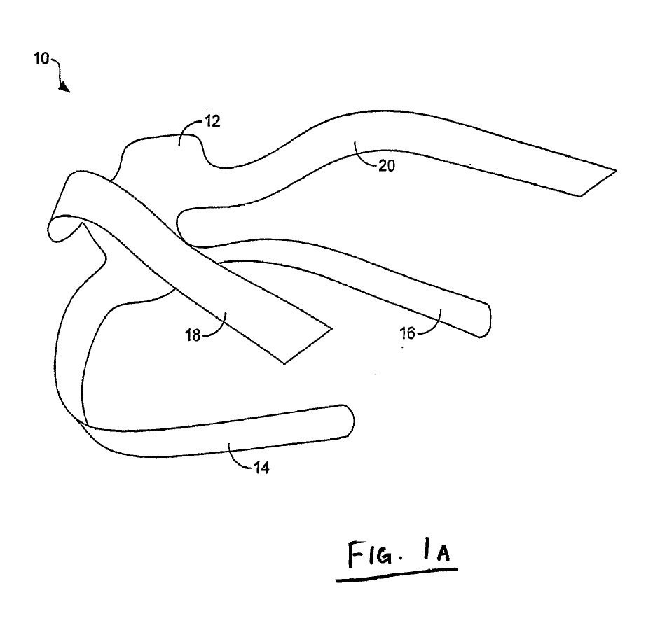

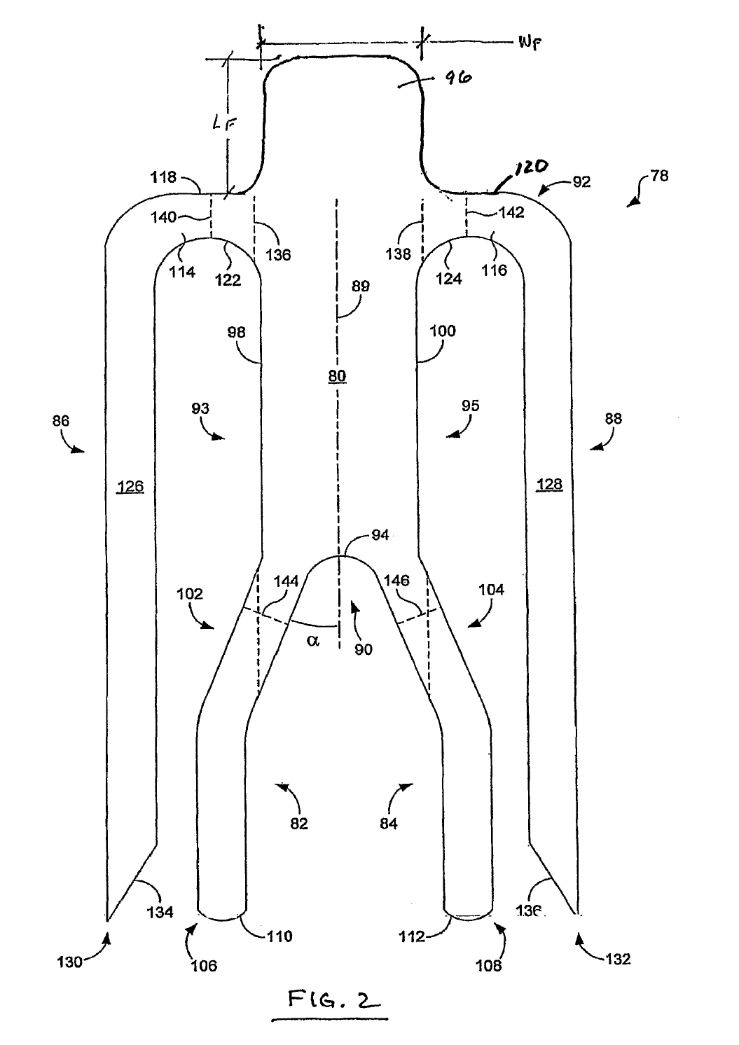

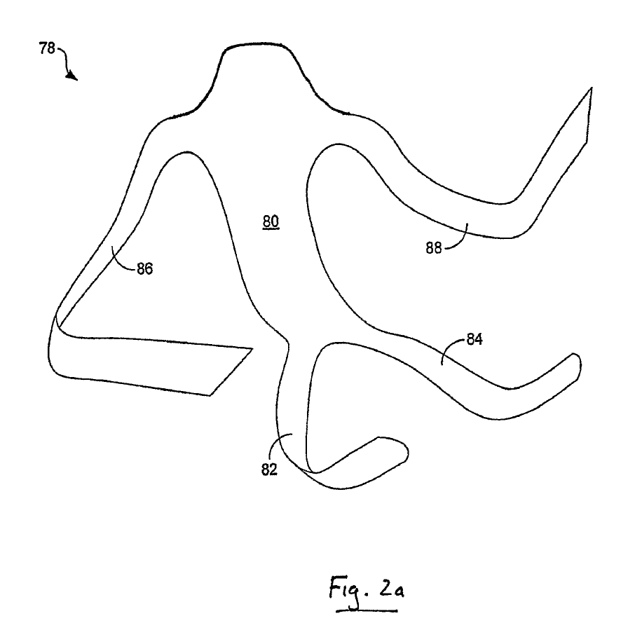

FIGS. 2 and 2A illustrate an embodiment of a posterior implant 78, which may be used to treat rectocele. As indicated in FIG. 2, the implant 78 generally comprises an elongated body portion 80, two front arms 82 and 84, and two rear arms 86 and 88. The posterior implant 78 is substantially symmetric about a longitudinal axis 89 that extends along the body portion 80. The body portion 80 is shaped and configured to be positioned between the vagina and the rectum and comprises a substantially rectangular shape defined by front side 90, a rear side 92, and opposed lateral sides 93 and 95. The front side 90 comprises a rounded edge 94 that extends inwardly towards the center of the body portion 80. The rear side 90 comprises an apical flap 96. In addition, the body portion 80 comprises substantially straight lateral edges 98 and 100 that, in some embodiments, are substantially parallel to each other. By way of example, the body portion 80 has a width dimension of approximately 40 mm and length dimension of approximately 100 mm.

The front arms 82, 84 each comprise two sections, first sections 102 and 104 that extend directly from the body portion 80, and second sections 106 and 108 that extend from the first sections 102 and 104. The first sections 102, 104 extend from the body portion 80 at divergent angles relative to the longitudinal axis 89. By way of example, each first section 102, 104 forms an angle, α, of approximately 20 degrees with the longitudinal axis 89. The second sections 106, 108 extend from the first sections 102, 104 in a direction that is substantially parallel to the longitudinal axis 89 and to each other. The second sections 106, 108, and the front arms 82, 84 terminate in rounded ends 110 and 112. By way of example, the front arms 106, 108 extend from the body portion 80 a distance of approximately 100 mm.

The rear arms 86, 88 extend from the rear side 92 of the body portion 80 past the front side 90 of the body portion to terminate adjacent the termination point of the front arms 106, 108. In the embodiment of FIG. 2, the rear arms 86, 88 initially extend laterally from the body portion 80 (substantially perpendicular to the longitudinal axis 89) along lateral sections 114 and 116. The lateral sections 114, 116 are defined by substantially straight rear edges 118 and 120 and rounded edges 122 and 124. Extending from the lateral sections 114, 116 are longitudinal sections 126 and 128 that are substantially parallel to the longitudinal axis 89 and to each other. The longitudinal sections 126, 128 terminate in pointed ends 130 and 132 that are defined by angled edges 134 and 136. In the embodiment of FIG. 2, the angled edges 134,136 diverge outwardly relative to the longitudinal axis 89. As with the pointed ends 60, 62 of the anterior implant 10 (FIG. 1), the pointed ends 130, 132 facilitate threading of the rear arms 86, 88 through a needle and aiding the surgeon in distinguishing the rear arms from the front arms 82, 84. By way of example, the rear arms 86, 88 span a distance (from the rear edge 96 of the body portion 80) of approximately 200 mm and are therefore approximately twice as long as the front arms 82, 84.

As with the anterior implant, the apical flap 96 is formed integral with the body 80 and is constructed to support the upper portion of the vagina. Also like with the anterior implant, portions or the entire apical flap may be cut off by the surgeon, as desired. In this regard, the surgeon can trim the implant so as to size it more appropriately to the patient's vaginal length. In one embodiment, the apical flap has a length L_{F} of approximately 40 mm and a width W_{F} of approximately 43 mm.

In some embodiments, like the anterior implant, all or a portion of the body portion 80 is formed of a relatively soft material as compared to the material of the arms 82, 84, 86, and 88 such that the portion of the implant 78 that is positioned between the vagina and the rectum is relatively soft and compliant while the arms are less compliant to ensure secure fixation and avoid implant migration and/or elongation. In the embodiment of FIG. 2, a central area bounded by dashed lines 136 and 138 (which extend from the rear side 92 past the front side 90 of the body portion 12 and along the front arms 82, 84) and having a width of approximately 43 mm comprises the relatively soft material. In addition, substantially the entire body portion 12, part of the rear arms 86, 88, and part of the front arms 82, 84 are provided with a layer of natural material. In the embodiment of FIG. 2, the extent of the layer of natural material is identified by the hidden lines 140, 142, 144, and 146, with the lines 140, 142 being substantially parallel to the longitudinal axis 89 and the lines 144, 146 being angled relative to the longitudinal axis.

FIG. 2A shows the posterior implant 78 in a perspective view in free space. The flexibility of the implant 78 is apparent in FIG. 2A from the illustrated orientation of the implant.

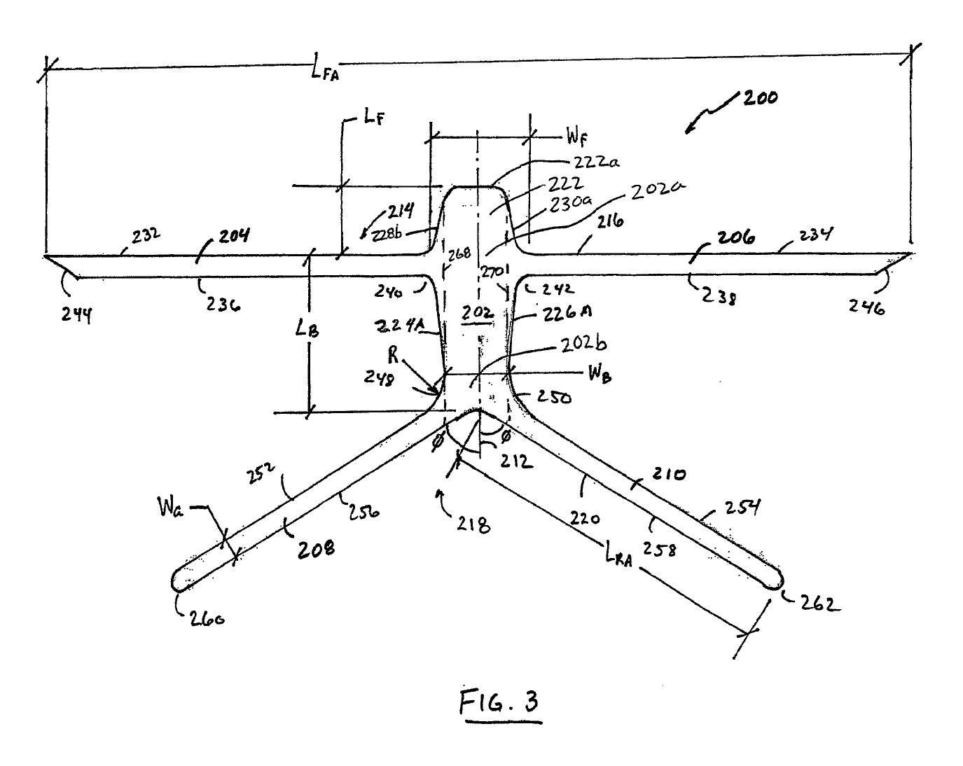

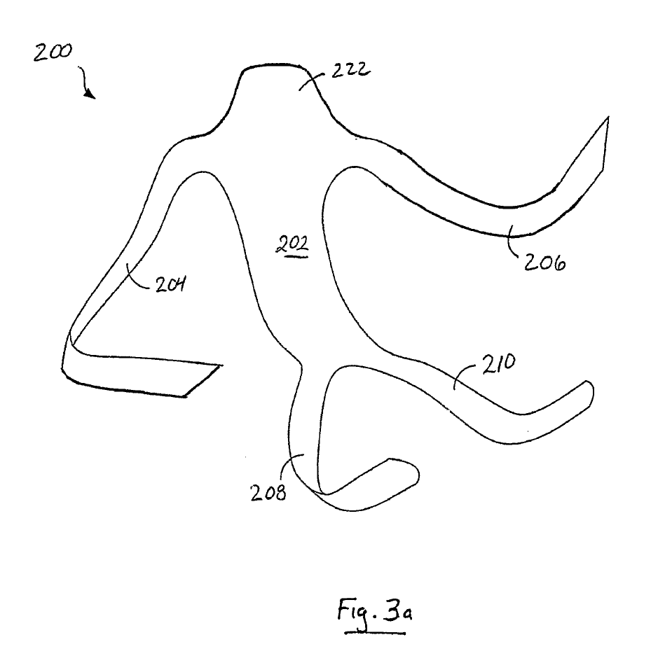

FIGS. 3 and 3A illustrate another embodiment of a posterior implant 200, which may be used to treat rectocele. As indicated in FIG. 3, the implant 200 generally comprises a body portion 202, two front arms 204 and 206, and two rear arms 208 and 210. The body portion 202 defines a central axis 212 about which the implant 200 is substantially symmetric and generally comprises a front side 214 defined by front edge 216 and a rear side 218 defined by a rear edge 220. In the illustrated embodiment, the front edge 216 is substantially straight and includes an apical flap 222 that extends from the body 202. The rear arms 208, 210 extend at an angle Φ relative to the central axis 212.

The body portion 202 includes a first end area 202A and a second end area 202B and the projection 222 has an end 222A. A pair of arms extends from the first end area and a pair of arms extends from the second end area. The body portion 202 further includes lateral edges 224A and 226. As is apparent from FIG. 3, the lateral edges 224A, 226A extend in a divergent manner from the first end area 202A to the second end area 202B of the body portion 202 (i.e., from the front arms 204, 206 to the rear arms 208, 210) at an angle relative to the central axis 212 such that the body portion 202 widens from the second end area 202B to the first end area 202A. The narrowed section, in one embodiment, approximates the narrowed perineal body in that the width is optimized for the width of the anatomy in this region of the pelvic floor. Lateral edges 228B, 230B of flap 222 extend in a divergent manner from the end 222A to the first end area 202A at an angle relative to the central axis 212 such that the flap 222 widens from the first end 222A to the first end area 202A. With the above-described configuration, the body portion 202 is shaped and configured to support the rectum with the flap 222 positioned relatively deep within the pelvis. In such a position, the rounded rear edges of the flap 222 are adapted to accommodate the contours of the pelvis interior. The apical flap 222 is constructed to support the upper portion of the vagina. Portions of the apical flap 222 can be trimmed or the entire apical flap can be removed by the surgeon, as desired, simply by cutting the mesh.

In this regard, the surgeon can trim the implant so as to size it more appropriately to the patient's vaginal length. By way of example, the body portion 202 has a length L_{B} (along the central axis 212) of approximately 90 mm and a width W_{B} at its narrowest section of approximately 40 mm. The rear flap 30 has a length L_{F} (along the central axis 212) of approximately 40 mm and the width W_{F} of the flap is approximately 60 mm-70 mm where it joins the body at the first end area 202A.

The front arms 204, 206 extend laterally from the body portion 202 substantially perpendicular to the central axis 212. In the illustrated embodiment, the front arms are substantially parallel to each other and comprise front edges 232 and 234. In addition, the front arms 204, 206 comprise rear edges 236 and 238 that are, in some embodiments, substantially parallel to the front edges 232, 234 and to each other. The rear edges 236, 238 join the body portion 202 at rounded inner comers 240 and 242. As is further depicted in FIG. 3, the front arms 204, 206 terminate with angled ends 244 and 246 that extend from the front edges 232, 234 of the arms 204, 206 to the rear edges 236, 238 of the arms. In the illustrated embodiment, the angled edges are angled inwardly from the front to the rear. As described below, the pointed ends both facilitate threading of the arms through an introducer needle as well as aid the surgeon in distinguishing the rear arms from the front arms. By way of example, the front arms 204, 206 span a length L_{FA} of approximately 580 Mm.

With continued reference to FIG. 3, the rear arms 208, 210 transition via a relatively large inside radius 248, 250 to thereby further reduce stress on the lower arms. In one embodiment the radius R is approximately 50 mm, however, the present invention is not limited in this respect, as other suitable radii may be employed.

In one embodiment, the rear arms extend at an angle from the body portion 202. In the embodiment of FIG. 3, the rear arms 208, 210 extend at an angle Φ of approximately 60 degrees relative to the central axis 212.

It should be appreciated that the present invention is not limited in this respect, as other suitable angles may be employed. Accordingly, angle Φ may be any angle in a range between approximately 30 degrees and 75 degrees. In one embodiment, the chosen angle follows the inherent diagonal lines of loops within the knit pattern of the mesh material, when mesh is employed as the support. In this regard, any tension on the arm results in pulling on the loops formed in the knit pattern in the tightest direction of the loops. As such, a maximum amount of tension can be applied to the arms without any undue stress that would otherwise result in premature failure, undue deformation and/or elongation, or inadequate performance of the implant.

Furthermore, the chosen angle, in one embodiment, more naturally approximates or follows the anatomical angle of the soft tissue through which the arms will extend so as to minimize any buckling or bunching of the arms as they pass through the soft tissue, thereby allowing the implant to lie in a more anatomically correct position/orientation.

Like the front arms, the rear arms 208, 210 comprise front edges 252 and 254 and rear edges 256 and 258. In some embodiments, the front edges 252, 254 and the rear edges 256,258 are substantially parallel. The ends 260 and 262 of the rear arms are rounded. By way of example, each rear arm spans a length L_{RA} of approximately 208 mm. Also, by way of example, each arm has a width WA of approximately 12 mm.

In some embodiments all or at least a portion of the body portion 202 is formed of a relatively soft material as compared to the relatively coarse material of the arms such that the portion of the implant 202 that supports the rectum is relatively soft and compliant while the arms are less compliant to ensure secure fixation in soft tissue and avoid implant migration and/or elongation. In the embodiment of FIG. 3, a central portion defined by the area bounded by dashed lines 268 and 270 having a distance between them of approximately 40 mm comprises the relatively soft material. In addition, as will be discussed below, the body portion 202, on one or both sides, can be provided with a natural layer of material.

FIG. 3A shows the posterior implant 200 in a perspective view in free space. The flexibility of the implant 200 is apparent from the illustrated orientation of the implant.

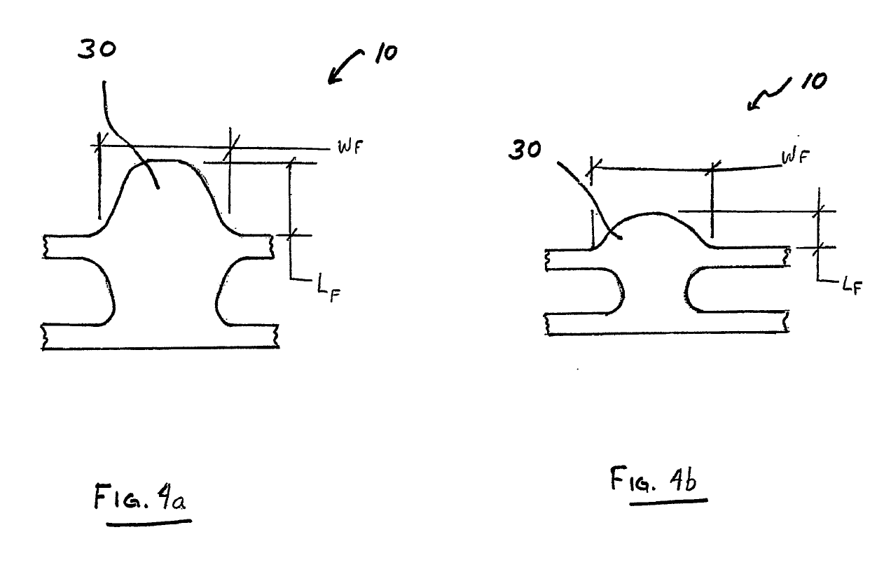

It should be appreciated that the shape of the apical flap may be configured differently, as the present invention is not limited in this regard. Accordingly, as shown in FIG. 4A, the apical flap 30 is shorter with a more rounded shape, such that the length L_{F} is approximately 30 mm. As shown in FIG. 4B, the apical flap is shorter and more rounded than even that shown in FIG. 4A, such that the length L_{F} is approximately 20 mm. Although the apical flap 30 is shown in FIGS. 4A and 4B as being on the anterior implants, such alternatives of the flap are applicable to the posterior implants. Further, as noted above, the surgeon may trim the apical flap to any desired dimension and/or shape.

Similarly, a portion of the body opposite the apical flap, whether for the anterior implant or the posterior implant, may be cut or contoured as desired by the surgeon to more readily approximate the anatomy of the patient. Thus, for illustrative purposes, as shown in posterior implant embodiment of FIG. 5, by trimming out material from portion 280 of the implant along, for example, dashed line 282, the arms are. effectively lengthened, whereas the body is effectively shortened.

In some embodiments, as shown in FIG. 5, at least two of the arms may include a tapered section 67 that taper to thinner ends 69. In this embodiment, the width W_{E} of the reduced width section is approximately 6 mm. Although the taper is shown on the arms having the angled ends, the taper may be included on (instead of or in addition to) the arms with the rounded ends.

To facilitate holding the arms on an insertion tool while the arms are being drawn through the soft tissue, as will be explained below the arms may be folded back on themselves to create a crease line. The folded arm thereby behaves like a hook to grasp onto the insertion tool. As shown in FIG. 7, which represents an exemplary arm of an implant, the end 284 of the arm 286 is folded back (as along arrow A in FIG. 7) to form a crease line 288. The crease line may be heat set to maintain its presence.

The crease line may be located in any suitable location, as the present invention is not limited in this respect. In one embodiment, the resulting crease line is spaced a distance F_{L} of approximately 50 mm from the end of the arm. In another embodiment, the distance F_{L} is approximately 40 mm. In the embodiment shown, the folded arm is shown to be the arm with the angled end. However, the arms with the rounded end may also include the fold. In one embodiment, the distance F_{L} for the arm with the angled end is approximately 50 mm, whereas distance F_{L} for the arm with the rounded end is approximately 40 mm.

The crease line may also aid the surgeon in determining how far to insert the arm through the introducer tool. In this regard, the surgeon may introduce the arm up to the crease line. However, the present invention is not limited in this respect, as other suitable indicators may be employed, such as colored bands, to indicate to the surgeon how far to insert the arm into the introducer needle.

As described above, the implant may include a natural material layer on one side of the implant. In one embodiment, the folded arm is such that the end of the arm 284 is folded toward the surface of the implant having the natural material layer 290, as shown in FIG. 7. It should be appreciated the present invention is not limited in this regard, as the arm may be folded away from the surface having the natural tissue layer.

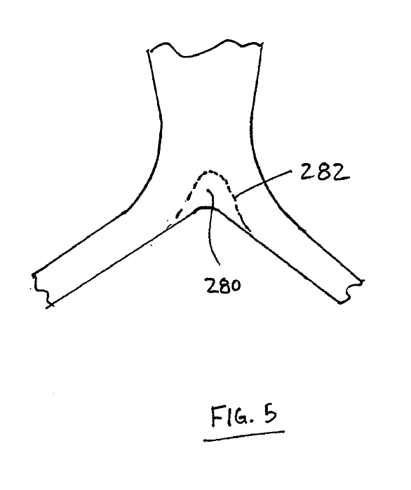

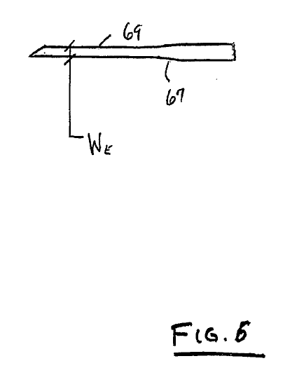

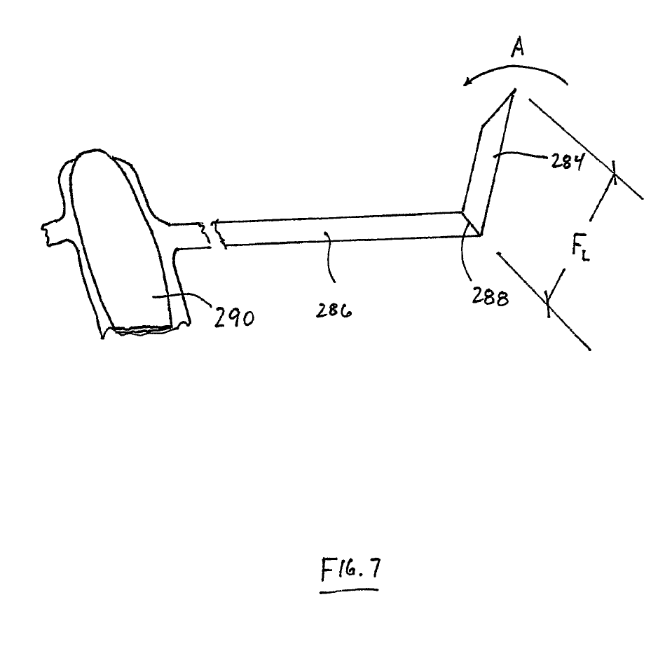

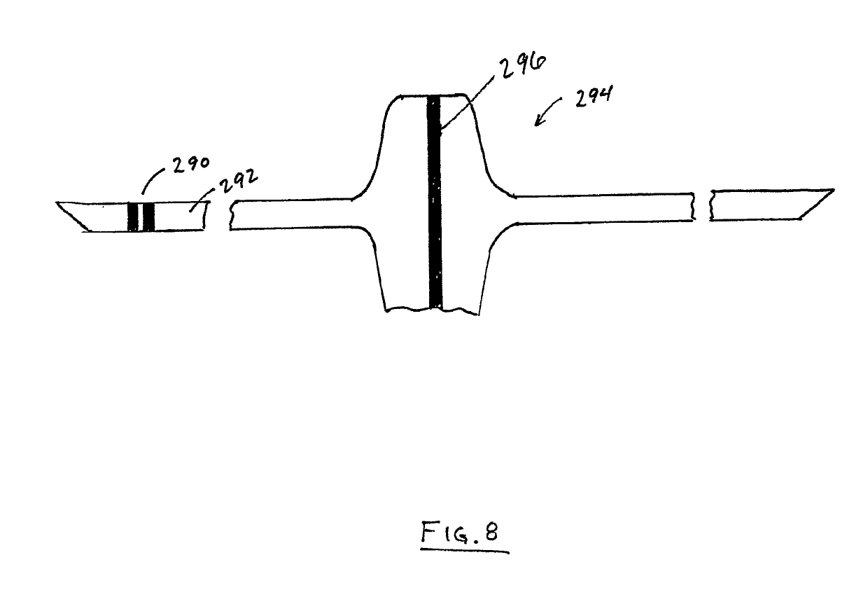

Also as described above, the angled arms may be used to distinguish the front from the rear arms. Similarly the implant may include one or more indicators to aid the surgeon in distinguishing the left side from the right side of the implant. The indicator(s) may be formed in any suitable manner, as the present invention is not limited in this respect. In one embodiment, the indicator is formed as a colored band or bands 290, as shown on the exemplary arm 292 of an exemplary implant 294 of FIG. 8. In the embodiment shown, the arm includes two colored bands on the left arm and none on the right arm.

Further, as shown in FIG. 8, the implant may include an indicator 296, which in this illustrative embodiment is also a color band, formed along the central axis of the body. In this way, the central indicator aids the surgeon in centering the implant. As mentioned above, the implant may include a natural layer over the body. In one embodiment, the central band is visible through the natural material layer or otherwise becomes visible once the natural material is re-hydrated at implantation.

The color bands may be formed in any suitable manner, as the present invention is not limited in this respect. In one embodiment, the color bands are added after the implant is formed by using a suitable ink or paint. In another embodiment, the color band results from a differently colored strand of the knitted mesh that is knitted in during knitting of an implant. Alternatively the color band may be formed as a separate element that is subsequently woven through the interstices of a mesh implant. In one embodiment, the color of the band is blue; however any other suitable color(s) may be employed, as the present invention is not limited in this respect.

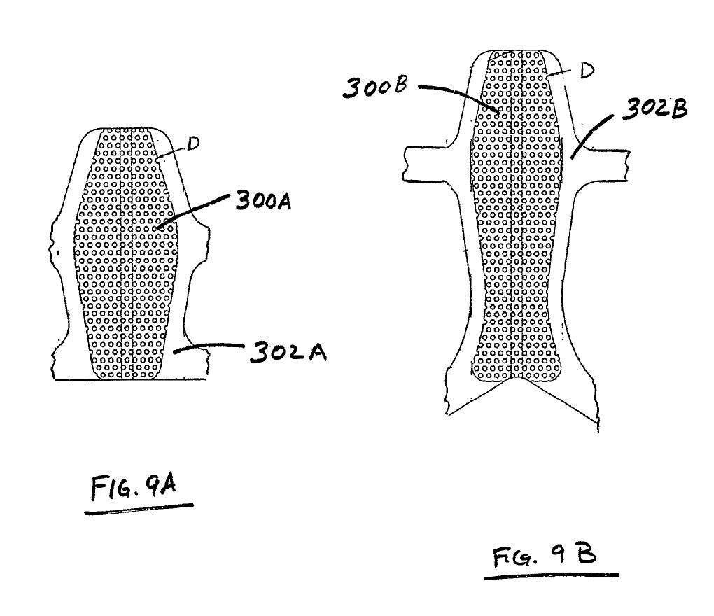

The implant may be formed with a synthetic layer and a natural layer over at least a portion of the synthetic layer. Referring to the embodiments shown in FIG. 9A and 9B, the implants are shown with a natural layer 300A, 300B disposed over the synthetic layer 302A, 302B over the central section and the apical flap of the implant. As shown, in one embodiment, the natural layer is spaced from the edge of the central section and the apical flap. In one exemplary embodiment, the natural layer is spaced a distance D in a range between approximately 3 mm and 7 approximately mm from the edge of the central section.

As will be discussed below, the implant is inserted through an incision in the vaginal mucosa. In this embodiment, the natural material extends along the incision line to protect the incision and reduce the likelihood of any erosion through the incision. As such, while the natural layer is spaced from the edges of the central section and apical flap along the sides thereof, in one embodiment, as shown, the natural layer extends the full length of the central section and apical flap to more fully protect the incision line. A certain amount of tolerance between the top and bottom edges of the synthetic material and the corresponding top and bottom edges of the natural layer may be acceptable. In one such example, a 2 mm mismatch may be acceptable.

In one embodiment, the natural layer(s) extends to the edge of the central section or, as shown in FIG. 9C, the natural layer(s) 300C slightly overlaps the central section and apical flap of the support layer 302C to prevent the edges of the support from being exposed to the host tissue. An overlap of the natural material of about 1 mm around the perimeter of the central area may be sufficient to provide adequate coverage of the support layer; however more or less overlap may be employed, as the present invention is not limited in this respect.

In another embodiment, the implant may be constructed with natural material only in the central section and apical flap and synthetic material only in the lateral sections. The implant may also be constructed with fill width synthetic strips that run under a portion of the central tissue section, providing a partial permanent support under the tissue for a more permanent repair. For instance, two full width mesh slings may be placed underneath and attached to a rectangular piece of natural material of about 5×8 cm in size.

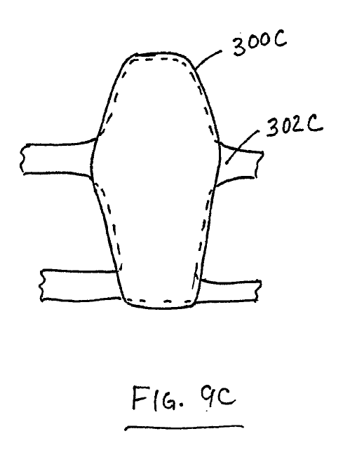

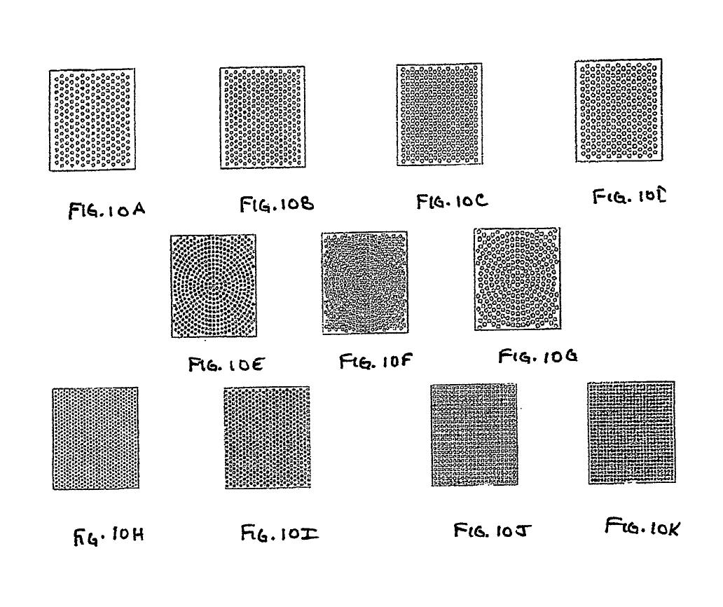

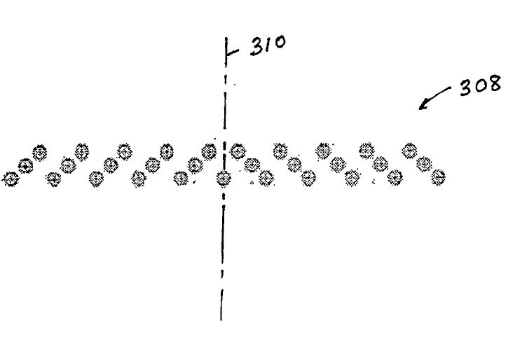

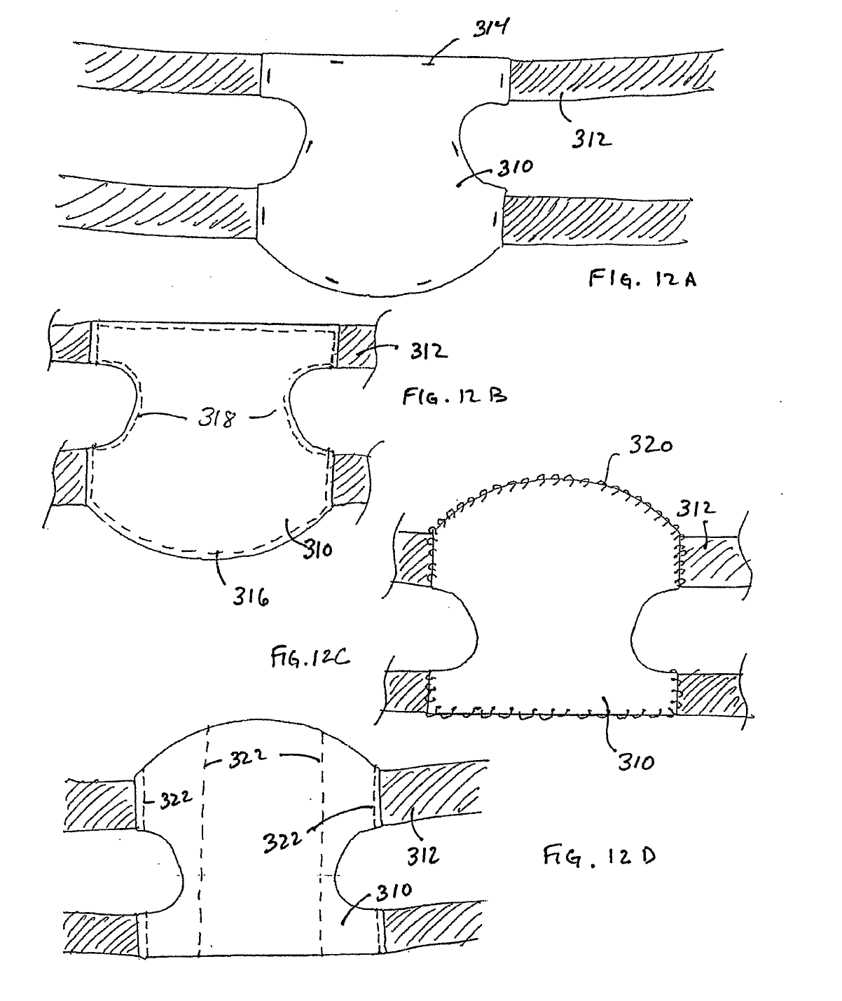

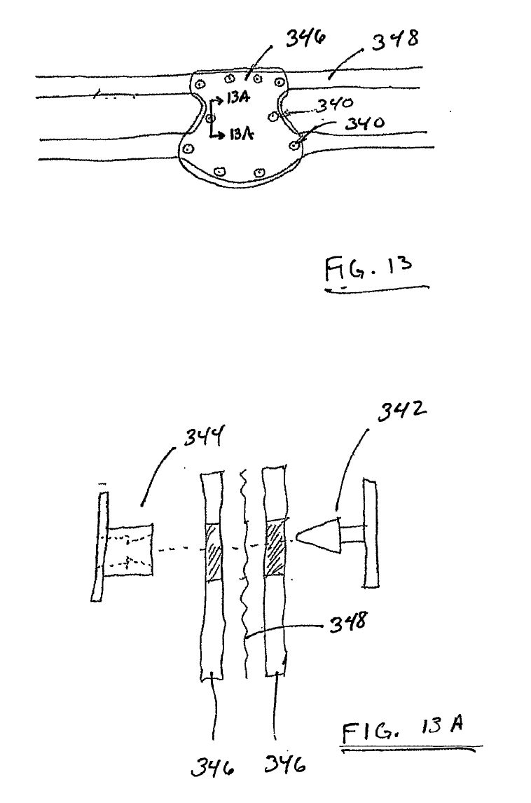

To improve the host tissue ingrowth through the natural material, various patterns of holes or slits may be used to enhance the porosity of the material. Round holes, square holes, oblong shaped holes, slits, or other shaped fenestrations may be employed to increase the porosity of the natural layer. By adding porosity, there is more surface area for host tissue contact and more area for improved revascularization and new tissue formation into the implant. By having holes in the natural material, there is also less risk of seroma or lo hematoma formation because the fluids can easily pass through and drain away from the implant. By removing material, the natural layer may become softer and more flexible. An exemplary hole size is about 2 mm in diameter, but hole sizes from 0.1 mm to 5 mm in diameter may be also be employed, as the present invention is not limited in this respect. Other suitable hole sizes may be employed, as the present invention is not limited in this respect. Further, any hole pattern and any number of holes per unit area may be employed, as the present invention is not limited in this respect. In some embodiments, the number of holes per unit area is high, whereas in other embodiments, the number of holes per area is low. In addition, the number of holes in a given layer of natural material may be consistent or may vary throughout the entire area of the natural layer, as the present invention is not limited in this respect. Also, any pattern resulting from the hole location may be employed, as the present invention is not limited in this respect. FIGS. 10A-10K represent exemplary hole sizes, patterns and number of holes per unit area, as indicated in the table below.

TABLE 1

| | Hole | Holes per | |
|---|---|---|---|
| Figure | Size (mm) | Area (h/cm²) | Pattern |
| 10A | 2.0 | 4.8 | Checkerboard |
| 10B | 2.0 | 6.2 | Checkerboard |
| 10C | 2.5 | 6.2 | Checkerboard |
| 10D | 2.0 | 4.8 | Checkerboard |
| 10E | 1.5 | — | Concentric Ring |
| 10F | 2.0 | — | Concentric Ring |
| 10G | 2.5 | — | Concentric Ring |
| 10H | 1.0 | 17.5 | Checkerboard |
| 10I | 1.5 | 12.4 | Checkerboard |
| 10J | 2.0 | 10.6 | Column/Row |
| 10K | 1.5 | 14.8 | Column/Row |

Additional illustrative combinations include embodiments where the hole size is 1.0 mm and the number of holes per area can any one or a combination of 28.1 h/cm², 34.7 h/cm² and 49.0 h/cm²; or the hole size is 1.5 mm and the number of holes per area can any one or a combination of 12.4 h/cm², 17.5 h/cm², and 22 h/cm².

In place of or in combination with the holes, a series of slits may be used to enhance the porosity and/or elasticity of the natural material. Slits are different from holes in that no material is removed from the base material. Slits are formed by making a series of multiple partial cuts or slices in the material that collectively act to allow flexing and elongation of the material.

It should be noted that each type of passage may provide certain benefits. In this regard, round or continuous cut holes minimizing the reduction in strength that can occur with slits, which can easily propagate a tear. On the other hand, slits may be beneficial because they act to soften the tissue and/or increase the elasticity of the tissue by expanding under tension, rendering the natural tissue more pliable and stretchable.

The holes or slits may also be arranged in an identifiable pattern that can be used to indicate preferential placement or orientation of the implant. For example, in an exemplary embodiment shown in FIG. 11, the hole pattern 308 may converge in a midline 310 at the center of the implant. Alternatively, a single row of holes may be left out on the centerline, indicating the center of the implant. This may assist surgeons with proper implant alignment and/or placement whereby the central section and apical flap of the implant is beneath the organ or other tissue to be supported. The holes and or slit pattern may also be positioned on the natural material in such a way so as to identify the left and right side of the implant, or the top and bottom of the implant, as the present invention is not limited in this respect. Such patterns may be used in addition to or instead of the colored bands mentioned above.

To join the natural tissue buffer layer(s) to the support layer, a variety of methods may be used. In an exemplary embodiment, a mechanical fixation is used, such as sewing or stitching the materials together. The sewing patterns may include tacking stitches or back stitches, continuous running stitches around the central graft, or any combination thereof. The threads can be secured via back stitching or hand/machine tying of the ends. FIGS. 12A-12D show various stitch patterns. In particular, in one embodiment, the natural material 310 may be attached to the support 312 by employing a single or double loop stitch 314, as represented in FIG. 12A. In one embodiment, the natural material 310 may be attached to the support 312 by employing a perimeter stitch 316, as represented in FIG. 12B, (a partial perimeter stitch may also be employed, for example, stitching may be absent from the center zone 318). In one embodiment, the natural material 310 may be attached to the support 312 by employing a circular stitch 320 over the edge of the natural material, as represented in FIG. 12C. In one embodiment, the natural material 310 may be attached to the support 312 by employing a line stitch 322 along desired lines of the natural material, as represented in FIG. 12D.

Alternatively or in addition to stitching, one or more mechanical rivets 340 may be used to pin the materials together, as shown in FIGS. 13 and 13A. Each rivet 340 includes a male pin 342 and a female receptacle 344 that snap together upon mechanical force through the natural layer(s) 346 and support layer 348. A hole may be preformed through one or both of the natural layer(s) and the mesh, as the present invention is not limited in this respect. Alternatively, the rivets may be thermal or ultrasonically welded, bonded via chemical means, or fused together using a variety of other suitable methods. Other methods of joining the materials may include staples, snaps, buttons, laser sintering, direct chemical bonding, and other methods known to those skilled in the art.

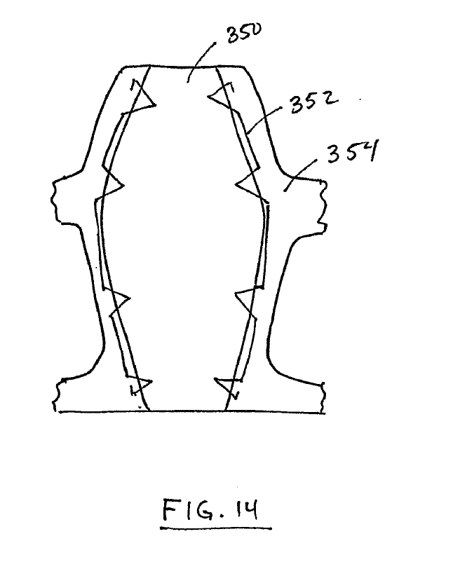

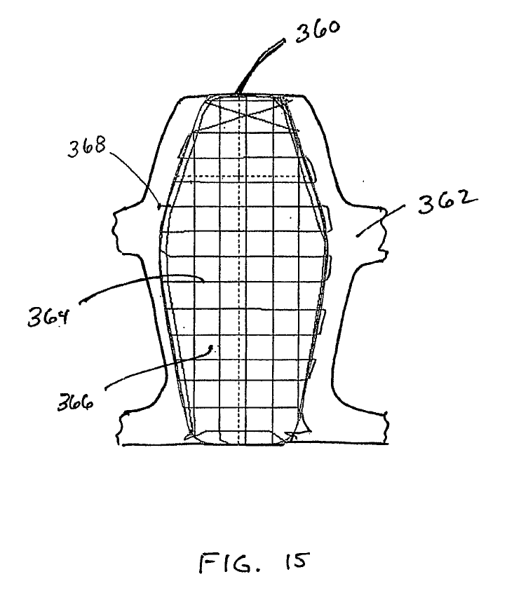

Prior to implantation, the implant is typically hydrated, causing the natural material to expand. Such expansion, if not controlled, can deform the implant rendering it unsuitable for implantation. In one embodiment, as shown in FIG. 14, the natural material 350 is stitched with stitch lines 352 in a manner whereby it is able to move or slide relative to the underlying support 354 along the length of the stitches. As a result, deformation of the implant is reduced or eliminated.

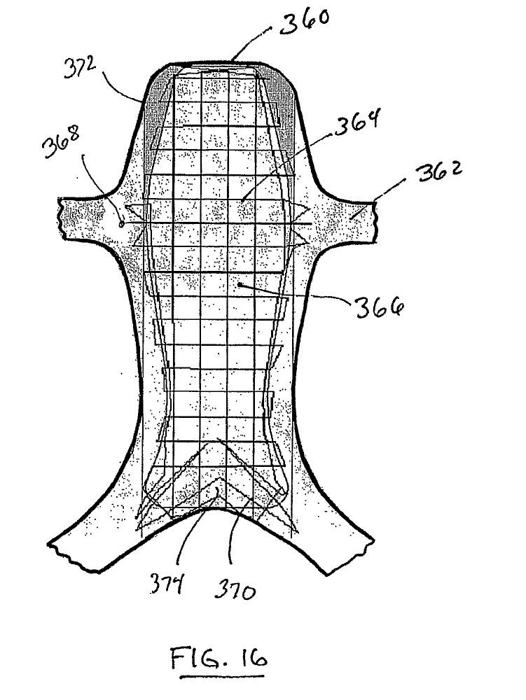

Alternatively, the natural layer may be more securely held down to limit movement. In the embodiments shown in FIGS. 15 and 16, the natural material 360 is held to the underlying support 362 with crisscrossing stitches 364, resulting in a checkerboard pattern. As a result, when the natural material is rehydrated during implantation, the portions not anchored in between the stitch lines (such as portions 366) expand and puff out, resembling a quilt.

In one embodiment, the spacing between the longitudinal and lateral stitch lines is optimized to limit the amount of movement and resulting curling of the implant upon rehydration, yet limit the amount of synthetic material of the stitch that the overlies the natural material. In one embodiment, the spacing between the longitudinal and lateral stitch lines is between approximately 5 mm and approximately 10 mm, although other separation distances may be employed, as the present invention is not limited in this respect. In one embodiment, the spacing is approximately 7 mm. Although the stitch lines are so spaced, each stitch line itself may pierce through the natural and support layer every approximately 3-4 mm. The stitching material may be any suitable material including absorbable or non-absorbable material, as the present invention is not limited in this respect.

The stitch may begin and end at any suitable location, as the present invention is not limited in this respect. In one embodiment, the stitch begins near the junction of the apical flap and the body, as shown by reference numeral 368.

The stitching may also be used to strengthen some areas of the implant. For example, tension on the arms may tend to deform the natural material and/or the synthetic material at that junction. Similarly, when the prosthesis is implanted by drawing the arms through tissue, as will be explained, the edge of the natural material near the arm may rub against the host tissue and curl back on itself Accordingly, as best shown in FIG. 16, additional stitch lines 370 are formed near the arms for added strength at this relatively high stress junction. In one embodiment, the stitch lines are angled relative to the checkerboard pattern to approximately follow the tension applied by the arms.

An additional benefit may be realized by reinforcing certain sections with additional stitching. When a surgeon desires to trim certain areas of the implant, for example, the tip of the apical flap 372 or the area 374 between the two angled arms, the surgeon will cut across the checkerboard patterned the stitch, potentially comprising the integrity of the fixation of the natural material. However, the surgeon is less likely to cut through the angled stitches, but rather would follow the angled stitch line, and the integrity of the attachment of the natural material is maintained to an acceptable level.

The overall thickness of the hybrid implant may be 1 mm or less, although other thicknesses may be used as well, as the present invention is not limited in this respect. In one embodiment where the support layer is sandwiched between the layers of natural material, the thickness of the hybrid implant is approximately 1 mm. It should be appreciated that if the implant is too thick, it may become too stiff for surgical use, or it may prevent sufficient ingrowth of host tissue, possible leading to erosion or rejection of the implant. The individual natural tissue layer(s), in one embodiment, is in the range of approximately 0.1-1.0 mm in thickness. In some embodiments, the thickness may be in the range of approximately 0.2-0.5 mm. When the natural material layer(s) is layered over the mesh, the tissue tends to compress and fill into the mesh pore interstitial spaces, creating a total hybrid implant thickness that is less than the sum of the parts. In this regard, in one embodiment, an implant having two layers of 0.5 mm thick natural material and a single mesh layer of about 0.4 mm thick, the total implant thickness is about 1.0-1.2 mm. In an alternative embodiment, the mesh may be covered with a single layer of a natural material, resulting in a thinner implant as compared to the sandwiched construct.

The natural tissue layer of the implant provides a more natural tissue buffer or interface between the patient's tissue and the implant, shielding the patient's tissue from the potentially more abrasive synthetic material. The natural material can be any suitable material, including, but not limited to biologically-derived materials, such as cadaveric (human) or xenograft tissue (particularly of porcine or bovine origin)—for example dermis processed to make an acellular collagen scaffold or intestinal submucosa or other biological material and/or bioengineered materials. Collagen materials can be obtained from various sources such as that available from Cook Biomedical, Inc. under the name COOK SURGISIS. In one embodiment, the natural material comprises a cross-linked porcine dermal collagen material, such as Pelvicol® surgical implant from Tissue Science 25 Laboratories plc, or Pelvisoft® acellular collagen biomesh produced by Sofradim. Other suitable bioengineered materials may be employed as the present invention is not limited in this respect.

To resist and extend the time for breakdown of the tissue, the tissue may be cross-linked to prevent the breakdown of the collagen by collagenase. Clinical studies have shown that most erosions occur in the first 12 months after implantation, so a natural material that can resist breakdown over this time period may be sufficient for preventing most erosions. However, the present invention is not limited in this regard as materials that breakdown in less than 12 months may be employed because they may still be present within the body, albeit with less strength. The level of crosslinking can range form being lightly crosslinked to heavily crosslinked, depending upon the desired time period after implantation for when the natural material breakdowns.

In another embodiment, the natural material may be ground up and processed into a gel that can be molded or extruded over the synthetic material. One benefit of this configuration is that no secondary attachment is necessary for joining the materials together. In one embodiment, the thickness of the overmolded layer of natural material completely encapsulates and shields the synthetic mesh from host tissue contact for a duration of at least 3 months post-implantation.

The synthetic material provides the permanent support for the overall implant. The synthetic material is formed into an ingrowth layer of tissue infiltratable fabric. The ingrowth layer is formed of a biologically compatible, flexible material that includes a plurality of interstices or openings which allow sufficient tissue or muscle ingrowth to secure the prosthesis to host tissue or muscle after implantation. In an exemplary embodiment, the synthetic material is a knitted, monofilament, polypropylene surgical mesh. Various knit constructions may be used, but an exemplary pore size is about 0.5 mm-2 mm, having a mesh density less than 60 g/mm². A chain stitch or inlay cross-fibers may be used to provide added stability to the base knit. The knit should provide a soft, pliable support for implantation. The knit should also be stable under tension, reducing the likelihood of any unraveling or curling particularly in the arms of the implant. In one embodiment, the implant material has isoelastic properties in both the length and width directions.

In addition to the above-noted BARD MESH, other materials that can be utilized include SOFT TISSUE PATCH (microporous ePTFE—available from W.L. Gore & Associates, Inc.); SURGIPRO (available from US Surgical, Inc.); TRELEX (available from Meadox Medical); PROLENE and MERSILENE (available from Ethicon, Inc.); and other mesh materials (e.g., available from Atrium Medical Corporation). It also is contemplated that the mesh fabric may be formed from multifilament yarns and that any suitable method, such as knitting, weaving, braiding, molding and the like, may be employed to form the prosthetic mesh material.

In one embodiment, as mentioned above, portions of the implant may be formed with a different knit construction than that employed in other portions of the implant. For example, the center of the implant may include a looser, more porous knit pattern resulting in a softer mesh area whereas the lateral segments may include a tighter, less porous knit pattern. One benefit is that a softer knit area may be used in the center for more compliant organ support, while the lateral sections can be made from a stronger knit to provide added strength.

It should be appreciated that the present invention is not limited in this regard, as the knit pattern may be uniform throughout the implant. Further, although the dual-knit pattern may be employed, the implant may include three or more knit patterns. Also, in one embodiment, the synthetic portion of the implant is formed as a single fabric, having one or more knit patterns; however, the implant can be constructed with one or more separate pieces (either having the same or different knit patterns) attached together.

In one embodiment, the lateral anchoring bands are formed with “meshing” sheets of partial weft to provide the dual knit configuration. As a result, greater stability can be achieved in the knit with the use of “meshing” lateral portions because the additional fibers being knitted overlap and intertwine with one another providing a more reinforced knit, as opposed to “non-meshing” sheets whose fibers do not intertwine with one another. Other mesh materials may be employed, as the present invention is not limited in this respect.

The mesh is not limited to being knitted from polypropylene fibers, as the present invention is not limited in this respect. Other materials that may be used include polyethelene, PTFE, polyester, or other materials suitable for implantation. The materials may be monofilament or multifilament yarn, or could even be extruded or molded into a single sheet, and preformed with holes to promote drainage and/or ingrowth. In addition, absorbable materials may also be used such as polylactic acid (PLA), polyglactin (VICRYL—available from Ethicon, Inc.) and polyglycolic acid (DEXON—available from US Surgical, Inc.) or other materials commonly used in absorbable surgical materials such as absorbable sutures. These materials can have their properties adjusted to prolong the degradation period over several years if needed.

The support layer can be cut to shape using a variety of methods, as the present invention is not limited in this respect. For example, steel rule dies or other cutting operations may be employed. When a mesh support layer is cut, the edges can fray, so methods such as heat staking the ends of the mesh or using ultrasonic cutting may be used to prevent the unraveling of the mesh after cutting. Certain knit constructions may be used to prevent unraveling, for example, employing 2 or more bar construction during the warp knitting process.

To further improve the stability and strength of the mesh under tension, a heated blade can be applied to the mesh to create a “welded band” where the mesh fibers are compressed and joined together to limit the slippage of fibers relative to one another. Various patterns may be used to create added strength in different directions, but may be especially useful for adding strength in the arm portions of the mesh. Alternatively, a secondary operation may be completed by sewing an additional thread into the arms of the mesh after knitting, or to reinforce the perimeter or central portion of the implant. With the additional thread, additional support is provided to reinforce the directions where the most tension will occur, such as during implantation when the arms are pulled into position through soft tissue.

In one embodiment, portions of the implant may include anchoring elements used to aid in holding the implant in place.

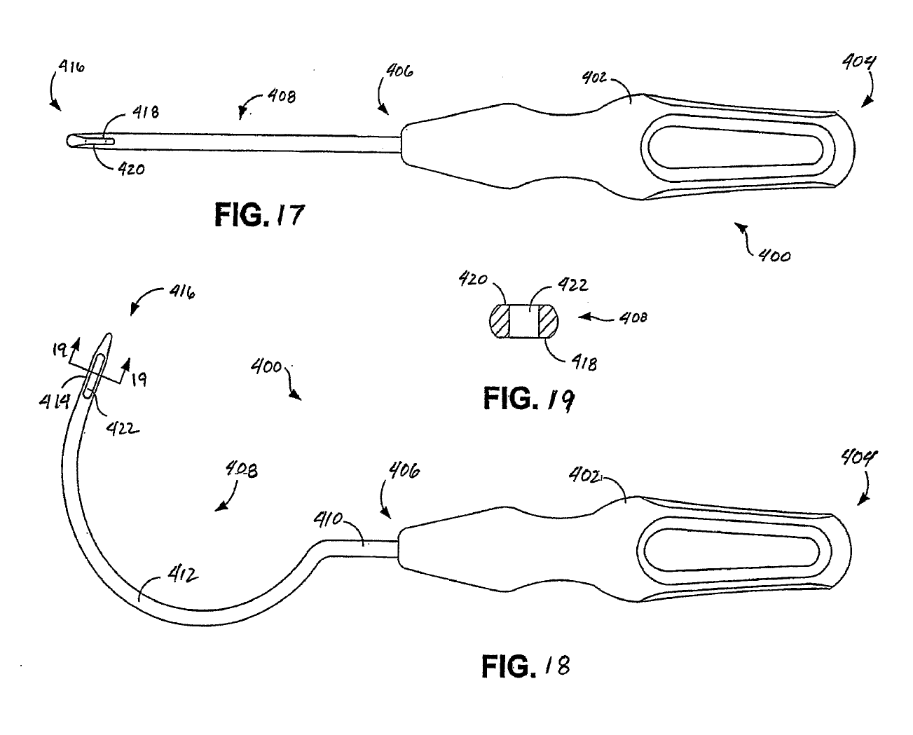

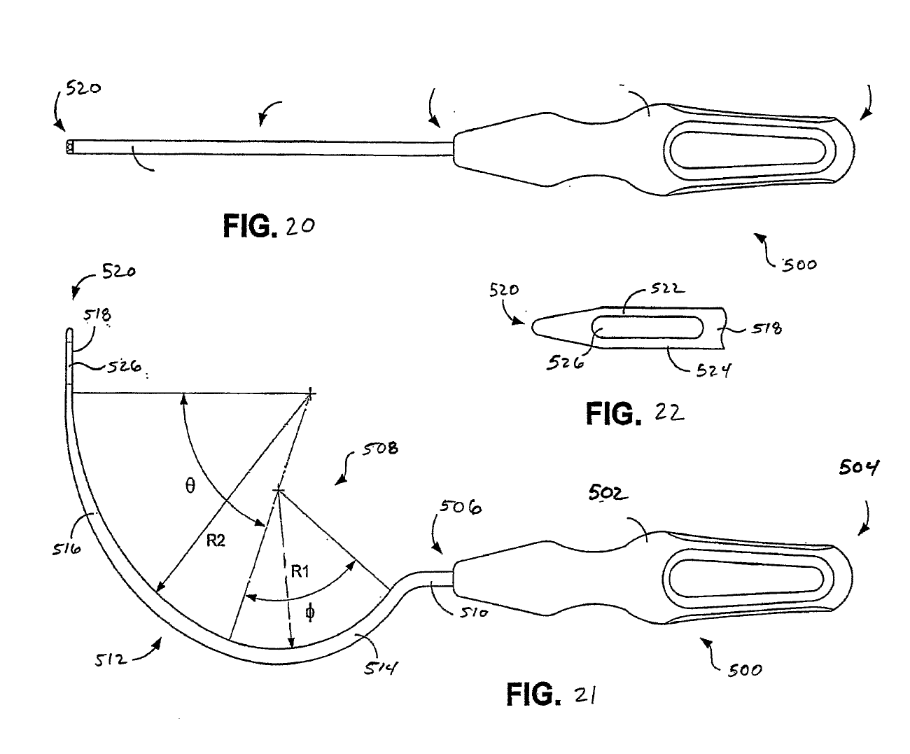

One example of a procedure to implant the prosthesis will now be described. FIGS. 17 and 18 illustrate an embodiment of an anterior introducer 400, which can be used to implant the anterior implant 10. As indicated in the figures, the introducer 400 comprises a handle 402 that includes a proximal end 404 and a distal end 406. The handle 402 is generally sized and shaped to fit within a surgeon's hand and, as depicted in FIGS. 17 and 18, can be contoured to facilitate firm gripping.

A needle 408 extends from the distal end 406 of the handle 402. As shown in FIG. 17, the needle 408 exists substantially within a single plane. As is most clearly shown in FIG. 18, the needle 408 comprises a first substantially straight section 410 that directly extends from the handle 402 substantially parallel to a longitudinal axis of the handle (not identified). Extending from the straight section is a curved section 412. By way of example, the curved section 412 extends along an arc having a radius of curvature of approximately 36.5 mm. The curved section 412 leads to a second substantially straight section 414 that terminates in a blunt tip 416. As is apparent from FIGS. 17 and 19, the straight section 414 is narrowed relative to the remainder of the needle 408 and is defined by two opposed lateral flat surfaces 418 and 420. Returning to FIG. 18, an elongated opening 422 is formed through the straight section 414 adjacent the tip 416. The opening generally extends along the length direction of the needle and serves as an attachment mechanism for releasably attaching an arm of an implant (e.g., anterior implant 10) to the needle. As shown in the cross-sectional view of FIG. 19, the opening 422 extends from one flat surface 418 to the other flat surface 420 of the needle. Although an opening has been identified, the needle can be provided with other attachment mechanisms that can secure the implant to the needle.

The handle can be constructed of any suitable rigid material, such as a metal or a polymeric material, as the present invention is not limited in this respect. The needle can be constructed of a biocompatible, strong material, such as stainless steel. In some embodiments, the handle and needle can be composed of the same material and may even be unitarily formed together so as to have a monolithic configuration.

FIGS. 20 and 21 illustrate an embodiment of a posterior introducer 500, which can be used to implant the posterior implant 78 (shown on FIG. 2) or the posterior implant 200 (shown in FIG. 3). As indicated in the figures, the introducer 500 comprises a handle 502 that includes a proximal end 504 and a distal end 506. The handle 502 is generally sized and shaped to fit within a surgeon's hand and, as depicted in FIGS. 20 and 21, can be contoured to facilitate firm gripping.

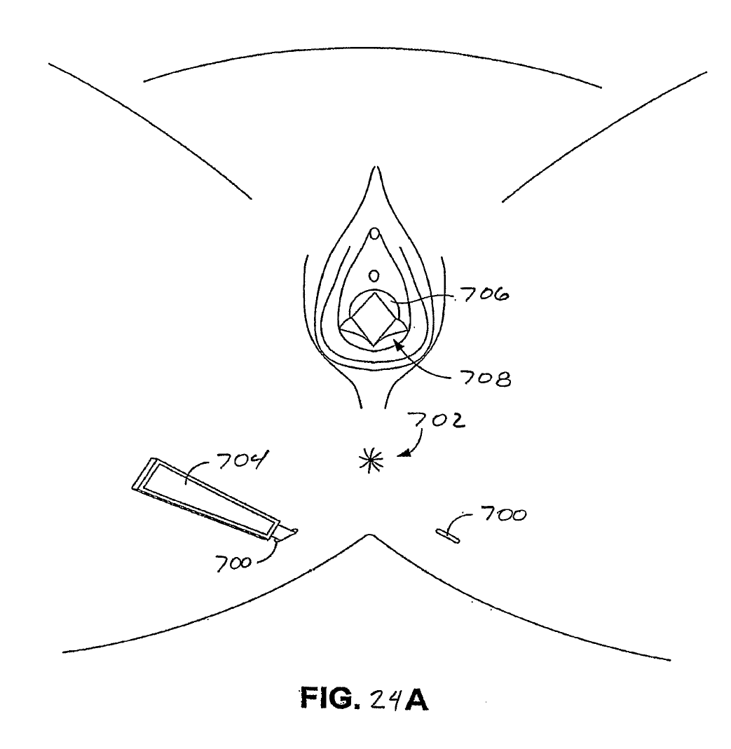

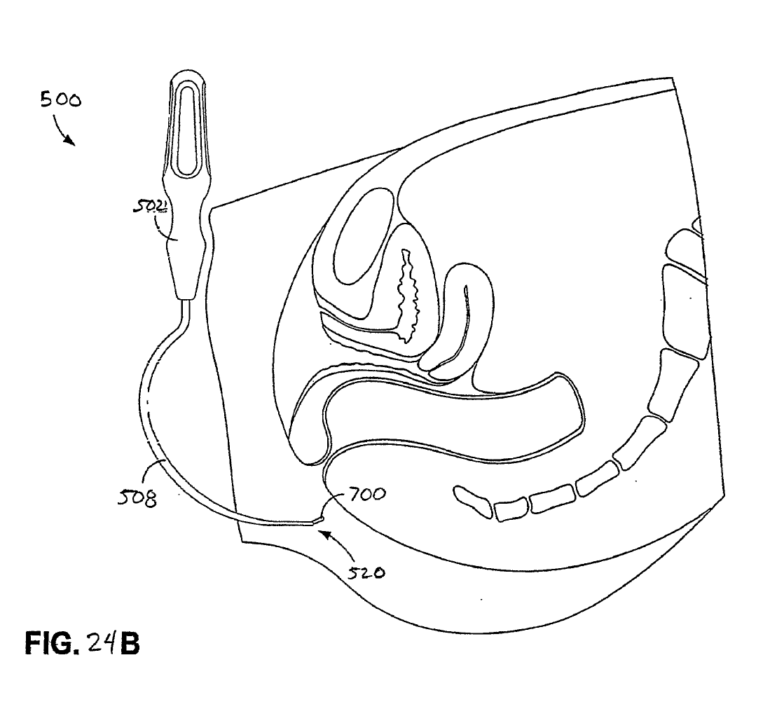

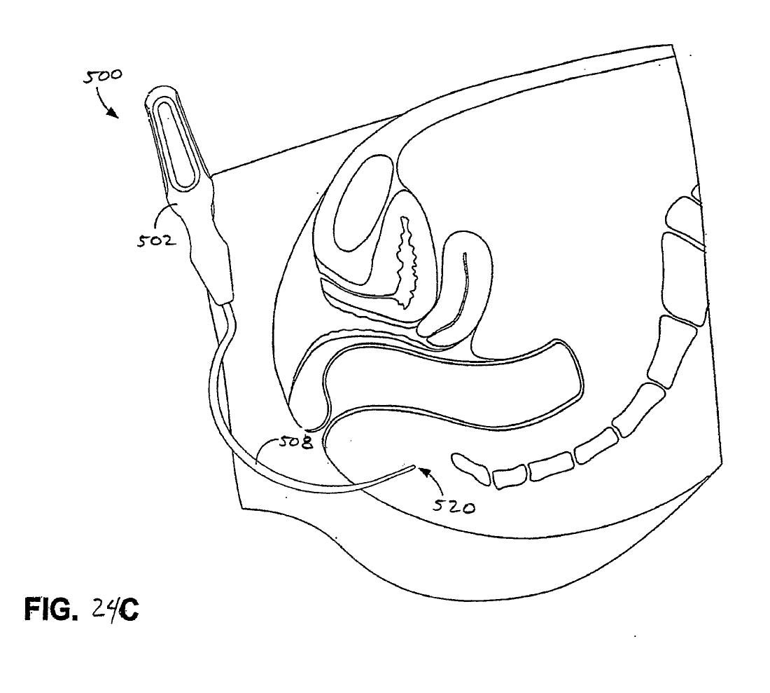

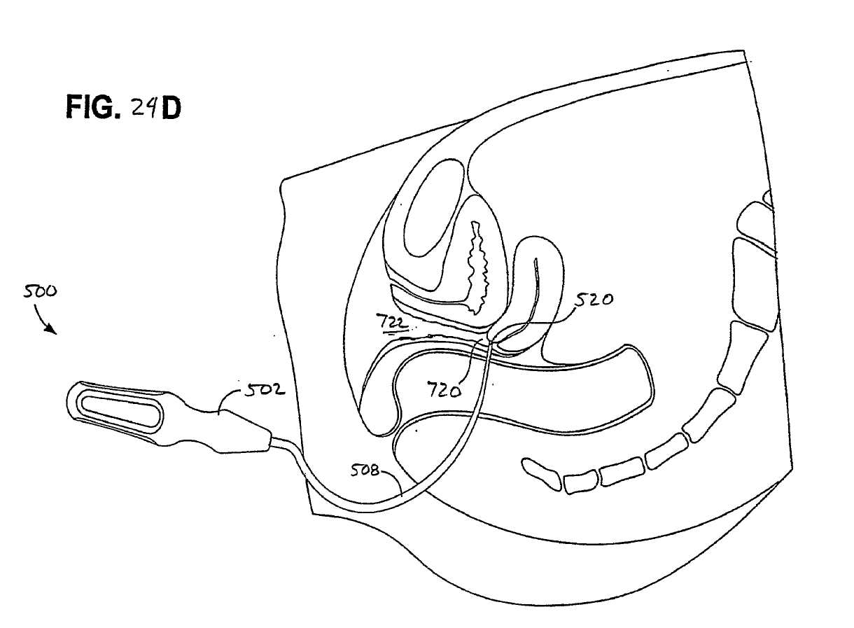

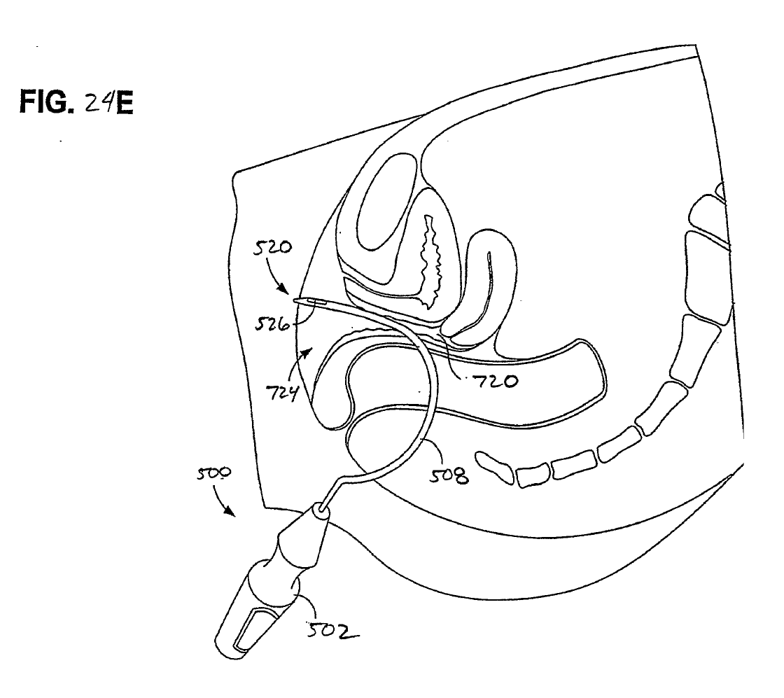

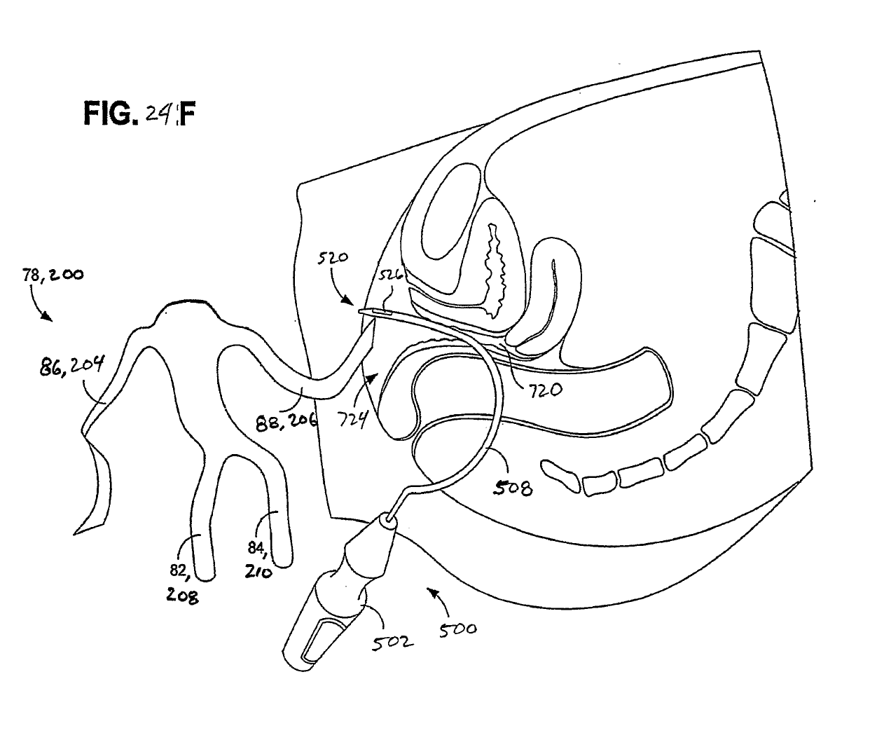

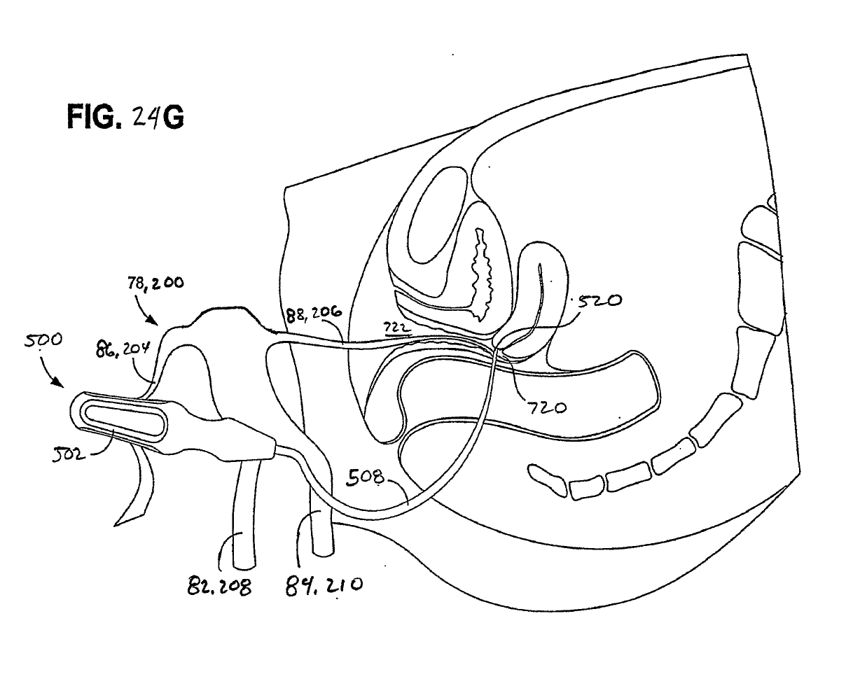

A needle 508 extends from the distal end 506 of the handle 502. As shown in FIG. 20, the needle 508 exists substantially within a single plane. As is most clearly shown in FIG. 21, the needle 508 comprises a first substantially straight section 510 that directly extends from the handle substantially parallel to a longitudinal axis of the handle (not identified). Extending from the straight section 510 is a curved section 512. In the illustrated embodiment, the curved section 512 comprises first curved portion 514 adjacent the straight portion 510 that has a relatively small radius of curvature, and a second curved portion 516 distant from the straight portion that has a relatively large radius of curvature. The two distinct portions 514, 516 of the curved section 512 facilitate manipulation of the needle 508 within the body such that the needle can be passed through an external incision, passed deep into the pelvic cavity adjacent the ischial spine, introduced into the vaginal vault, passed down through the vagina, and exteriorized from the vaginal introitus. More particularly, as is apparent from FIGS. 24A-24E described below, the second curved portion 516 having the greater radius of curvature is used to form a relatively straight passage deep into the pelvic cavity and to traverse the distance from the vaginal vault to the vaginal introitus, while the first curved portion 514 having the smaller radius of curvature is used to rotate or pivot the second curved portion to enable its traversal through the pelvis. By way of example, the first curved portion 514 has a radius of curvature R1 of approximately 48 mm through an angle of approximately 72 degrees, while the second curved portion 516 has a radius of curvature R2 of approximately 78 mm through an angle of approximately 70 degrees, such that the second curved portion has a radius of curvature that is nearly twice that of the radius of curvature of the first curved portion.

Extending from the second curved portion 516 is a second substantially straight section 518 that is substantially perpendicular to the first substantially straight section 510 and that terminates in a blunt tip 520. As is most clearly shown in the detail view of FIG. 22, the straight section 518 comprises substantially flat opposed edges 522 and 524. Between those edges is an elongated opening 526 that serves as an attachment mechanism for releasably attaching an arm of an implant (e.g., the posterior implant 78, 200) to the needle 508. Notably, while the opening 422 of the anterior introducer 400 may be said to extend laterally through the needle 408, the opening 526 of the posterior introducer 500 extends proximally-to-distally through the needle 500. Although an opening has been identified, the needle 500 can be provided with other attachment mechanisms that can secure the implant to the needle, as the present invention is not limited in this respect.

The handle 502 can be constructed of any suitable rigid material, such as a metal or a polymeric material, as the present invention is not limited in this respect. The needle 508 can be constructed of a biocompatible, strong material, such as stainless steel. In some embodiments, the handle 502 and needle 508 can be composed of the same material and may even be unitarily formed together so as to have a monolithic configuration.

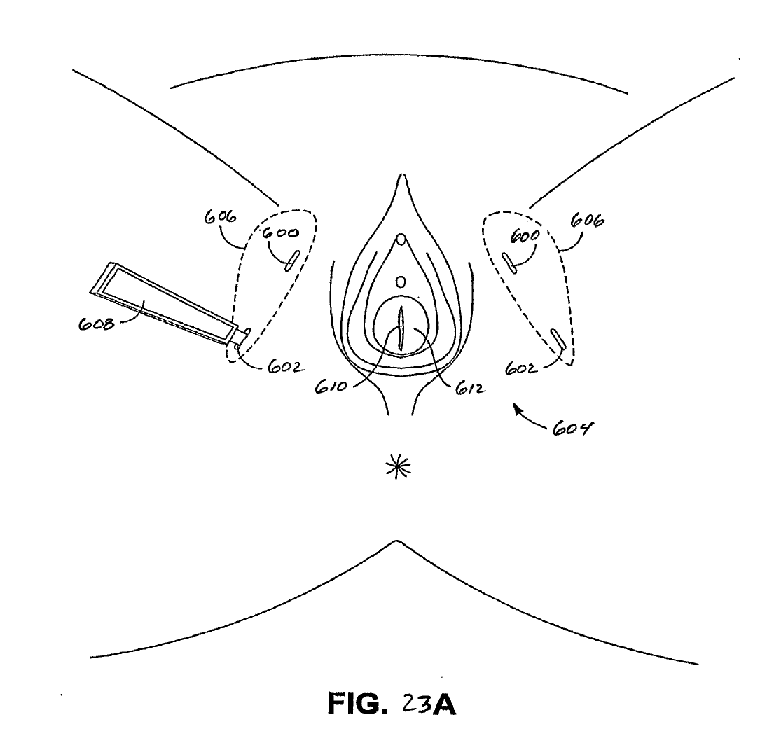

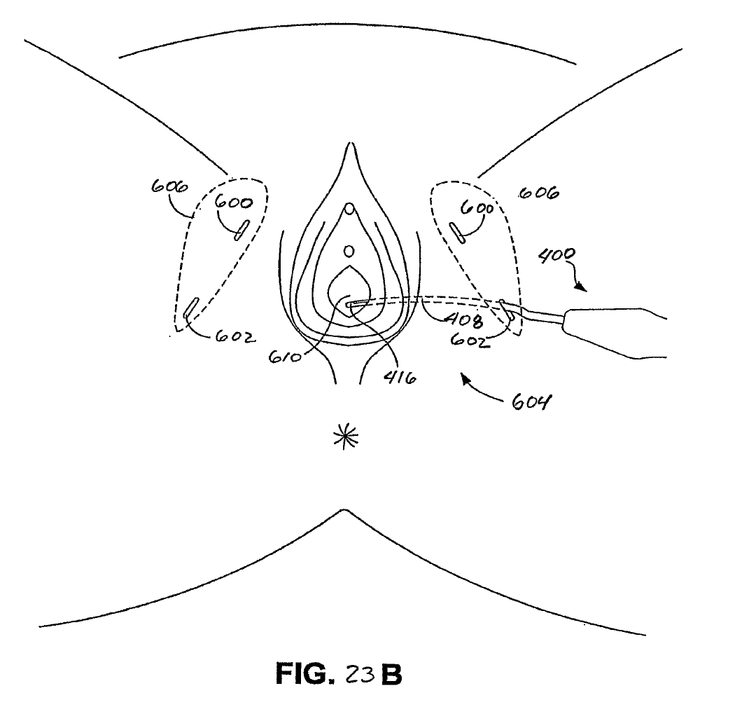

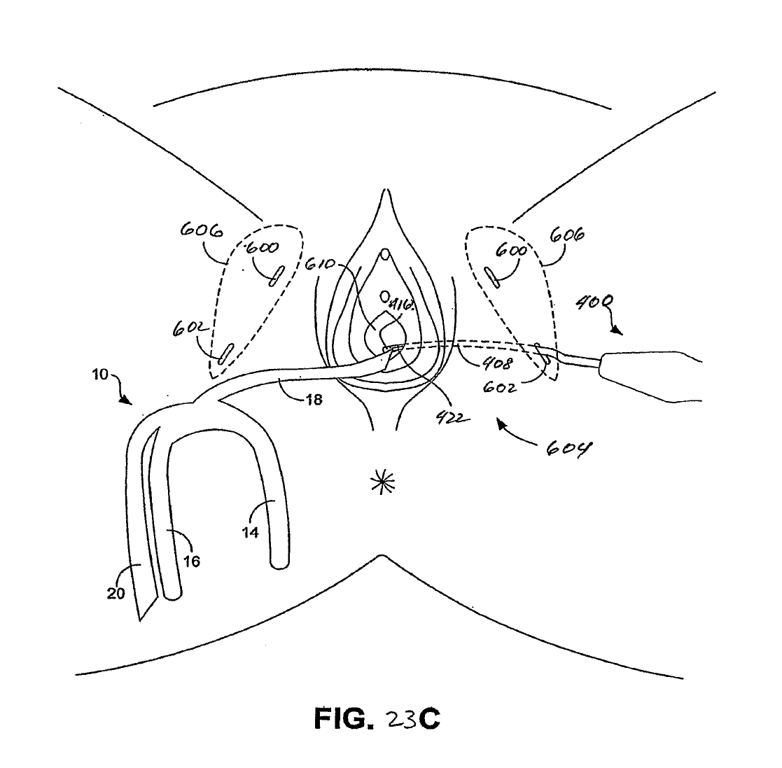

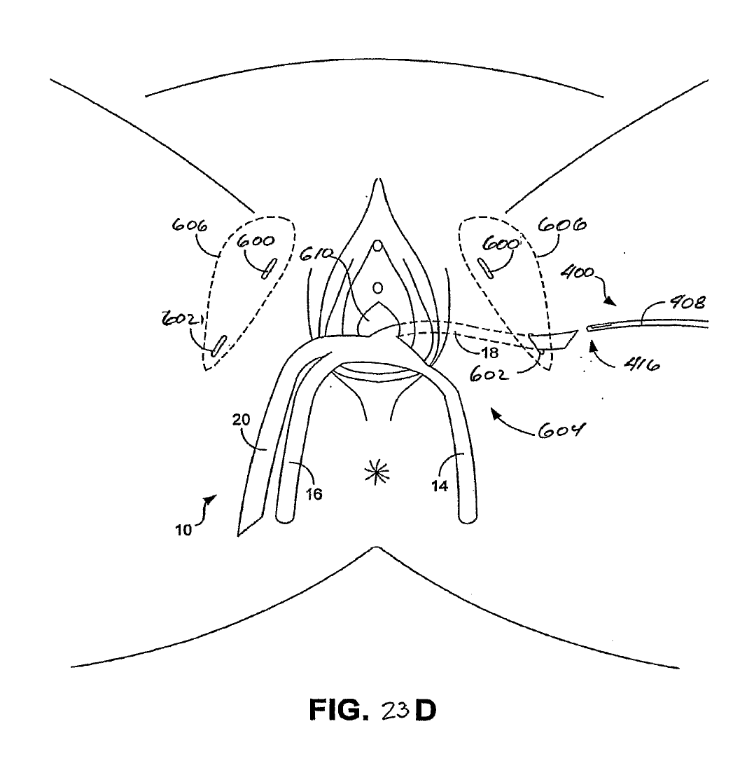

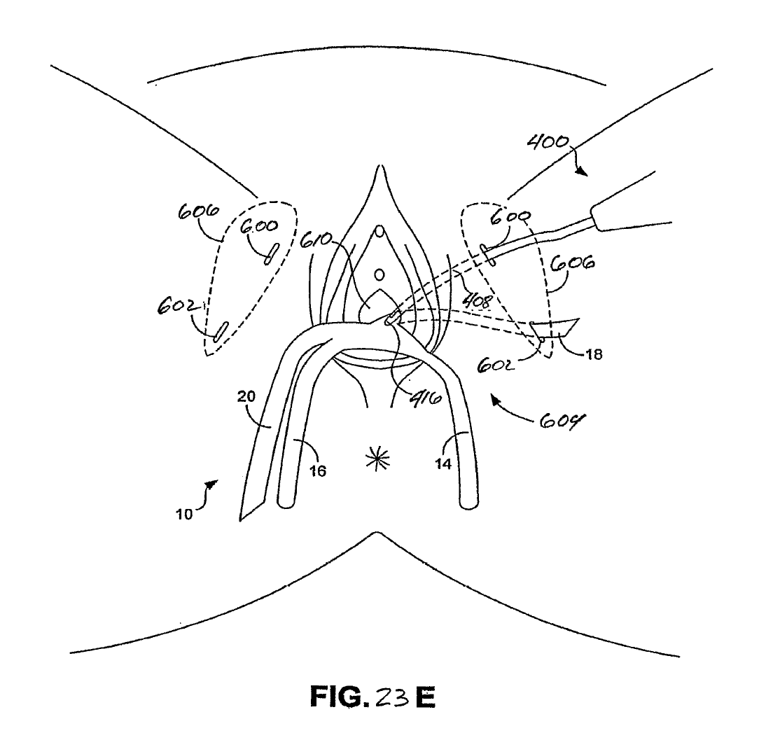

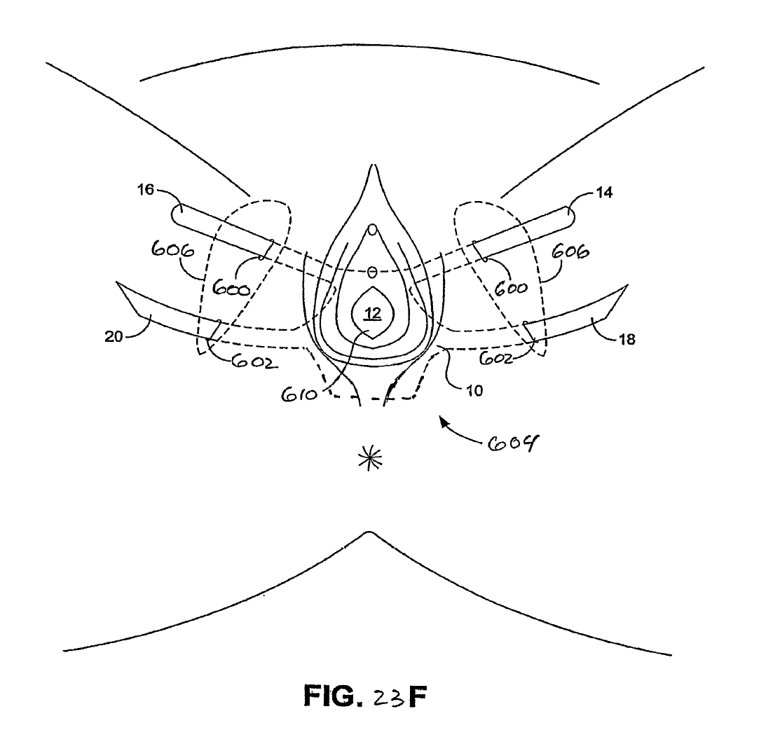

FIGS. 23A-23F illustrate an embodiment of a method for implanting a pelvic implant. More particularly, FIGS. 23A-23F illustrate a procedure for implanting the anterior implant 10 between the vagina and the bladder using the anterior introducer 400. Beginning with FIG. 23A, superior and inferior incisions 600 and 602 are made in the paravaginal region 604 in alignment with the obturator foramina 606 of the pubic bone (not shown). Those incisions can be made with a sharp device, such as a scalpel 608. In addition, a midline incision 610 can be made in the anterior vaginal wall 612 to provide access to the space between the vagina and the bladder. That space can then be separated by blunt and/or sharp dissection.

Turning to FIG. 23B, the needle 416 of the anterior introducer 400 is passed through one of the inferior incisions 602 and through the soft tissue of the pelvis until the tip 416 emerges from the vaginal incision 610 to become visible to the surgeon. Referring to FIG. 23C, the anterior implant 10 can then be associated with the needle 408. More particularly, one of the arms of the implant (e.g., rear arm 18) is passed through the opening 422 formed in the needle 408 adjacent the needle tip 416. If the implant includes an arm fold, the arm is inserted through the opening up to the crease. The arm fold facilitates “hooking” the arm to the needle. Notably, the surgeon can easily distinguish between the rear arms and the front arms of the implant 10 due to the pointed tips of the rear arms.

Once the implant arm 18 is associated with the needle 408, the needle may be drawback through the passage it formed pulling in the implant arm in tow until the needle exits the body and the implant arm occupies the passage and extends out from the inferior incision 602, as indicated in FIG. 23D.

At that point, one of the front arms (e.g., arm 14) can be implanted. This is accomplished by passing the needle 400 through one the superior incisions 600, for example the incision adjacent the inferior incision 602 through which the rear arm 18 was passed, until the needle tip 416 emerges from the vaginal incision 610, as indicated in FIG. 23E. The arm 14 can be associated with the needle 408 in similar manner to that described above by passing the arm through the needle opening 422 up to the arm fold, if included. The arm 14 can then be drawn through the passage formed by the needle 400 in similar manner to that used to draw the rear arm 18 through its passage (not shown).

The front and rear arms (e.g., arms 16 and 20) on the opposite side of the implant 10 can then be positioned in similar manner to the methods described above. One or more of the arms can then be tensioned as required to pull the body portion 12 into position between the vagina and the bladder, as indicated in FIG. 23F. At that point, the portions of the arms that extend beyond their respective incisions can be trimmed, the vaginal incision 610 can be closed, and the various external incisions can be closed.

In one embodiment, the long arms of the implant pass above the ischial spine and exit the inferior medial portion of the obturator membrane. The shorter distal arms enter beneath the bladder neck and pass laterally approximately through the superior medial portion of the obturator membrane out through the thigh incision over the obturator membrane.

FIGS. 24A-24L illustrate a further embodiment of a method for implanting a pelvic implant. More particularly, FIGS. 24A-24L illustrate a procedure for implanting the posterior implant (such as posterior implant 78 or posterior implant 200) between the vagina and the rectum using the posterior introducer 500. Beginning with FIG. 24A, small pararectal incisions 700 are made on either side of the anus 702 with a sharp device, such as a scalpel 704. By way of example, the incisions 700 are made approximately 2-3 centimeters (cm) posterior and lateral to the anus 702. In addition, a midline incision is made in the posterior vaginal wall 706 to form an opening 708 that extends from the vaginal introitus to the vaginal apex to provide access to the space between the vagina and the rectum. The vaginal mucosa may then be dissected away from the rectum using blunt and/or sharp dissection.

Turning to FIG. 24B, the tip 520 of the posterior introducer needle 508 is positioned at one of the incisions 700 with the introducer 500 oriented so that the handle 506 is generally vertical. Referring next to FIG. 24C, the introducer needle 508 is passed through the incision 700 and through the soft tissue of the pelvis toward the ischial spine (not identified). As the needle passes through the soft tissue, the introducer is rotated so that the handle 506 approximates a generally horizontal orientation, as indicated in FIG. 24D. As is further shown in FIG. 24D, the needle tip 520 is advanced through the posterior vaginal wall and into the vaginal vault 720 such that the tip is positioned within the vagina 722. That process can be aided by placing a finger (not shown) within the vagina to guide the needle tip into position.

With reference next to FIG. 24E, the introducer handle 502 is pivoted downwardly and rotated laterally relative to the patient to bring the tip 520 of the needle to the vaginal introitus 724 such that the needle tip and the opening 526 adjacent the tip are visible and accessible to the surgeon.

Referring next to FIG. 24F, one of the longitudinal arms (e.g., arm 88, 204) of the posterior implant 78, 200, respectively, is associated with the needle 508. In particular, the arm 88, 204 is passed through the opening 526 of the needle 508. If the implant includes an arm fold, the arm is inserted through the opening up to the crease. The arm fold facilitates “hooking” the arm to the needle. Again, the surgeon can distinguish the rear arms from the front arms due to the pointed tips of the rear arms.

Turning to FIG. 24G, the needle tip 520 can be retracted along the vagina 722 to the vaginal vault 720 to draw the implant arm 88, 204 back toward the vaginal vault. Referring to FIG. 24H, the implant arm 88, 204 can further be drawn through the passage formed by the needle 508 until the arm extends through the pelvis and out through the pararectal incision 700. At that point, the implant arm 88, 204 can be disassociated from the needle 508.

Next, as indicated in FIG. 241 the needle 508 can be reintroduced through the same pararectal incision 700 from which the implant arm 88, 204 extends in order to position the front arm (e.g., arm 84 of implant 78 or arm 210 of implant 200) on the same side of the implant as the implanted rear arm. As indicated in FIG. 24J, the needle tip 520 is passed along a relatively shallow path through the soft tissue of the pelvis until it emerges from the vaginal introitus 724. At that point, the front arm 84, 210 can be associated with the needle 508 by passing the arm through the opening 526 of the needle up to the fold, if included, and the arm can be drawn through the pelvis and out through the incision 700 in similar manner to the rear arm 88, 204. Similar procedures may then be performed to implant the arms (e.g., arms 82, 86 of implant 78 or arms 204, 208 of implant 200) on the opposite side of the implant on the opposite side of the vagina.

FIG. 24K illustrates the result after each arm has been implanted. As can be appreciated from that figure, the rear arms (only arm 88, 204 visible) are positioned relatively deeply and the front arms (only arm 84, 210 visible) are positioned relatively shallowly within the pelvis, and the body portion 80, 202 of the implant is positioned between the vagina 722 and the rectum 726. At that point, the excess portions of the arms can then be trimmed, the vaginal incision can be closed, and the external incisions 700 can be closed.

In one embodiment, the long arms of the posterior implant pass in front of or distal to the sacrospinous ligament at approximately the level of the ischial spine or alternatively at the surgeon's discretion pass through the ligament at approximately the level of the ischial spine and exits through the two pararectal incisions. The short arms pass through the posterior floor lateral to the sphincter and out the same pararectal incisions.

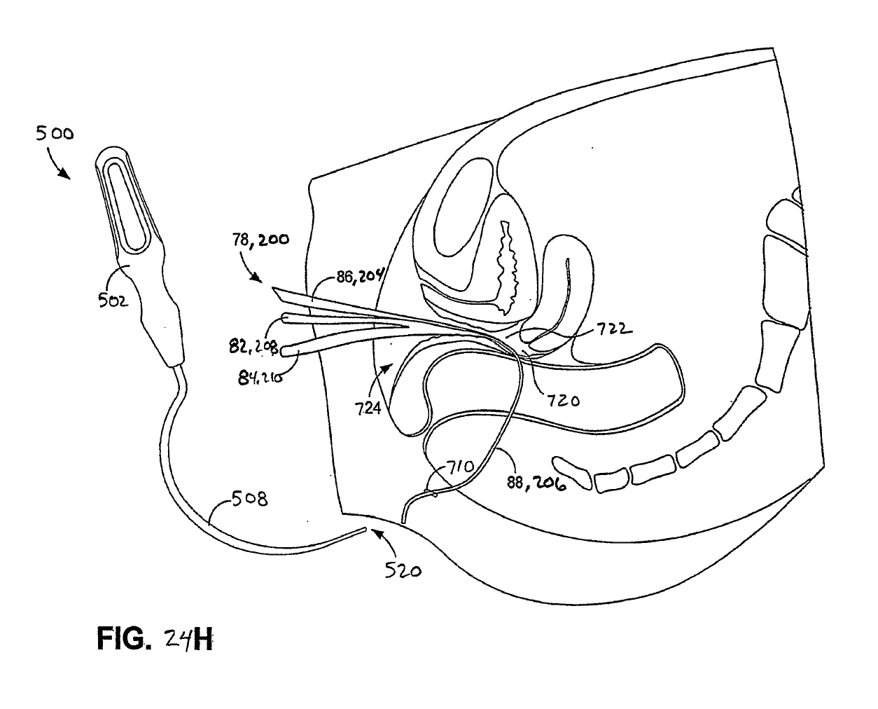

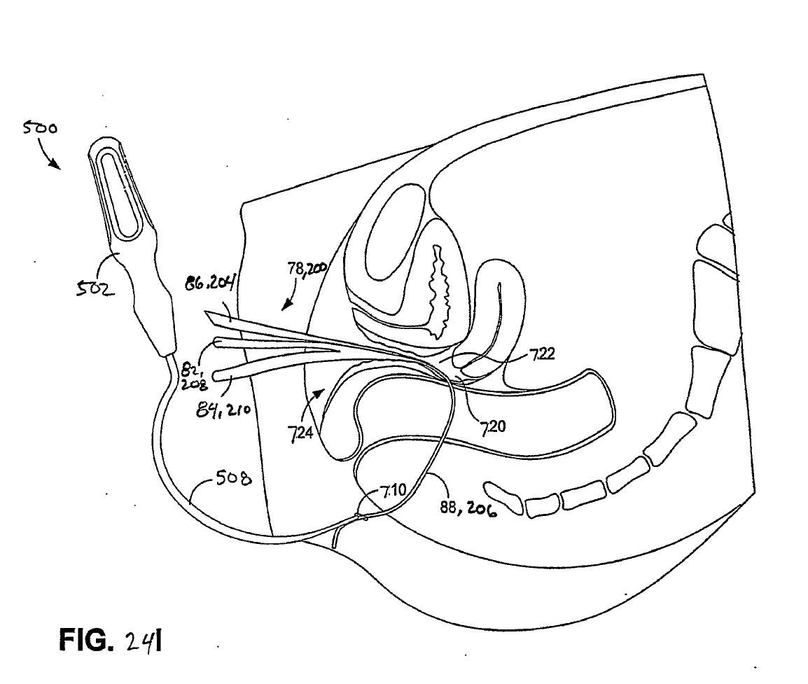

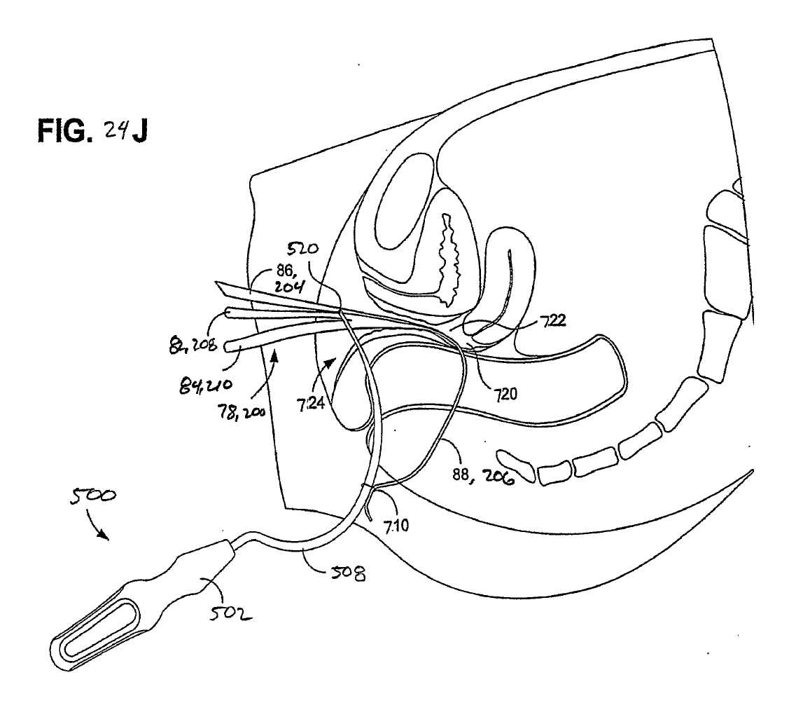

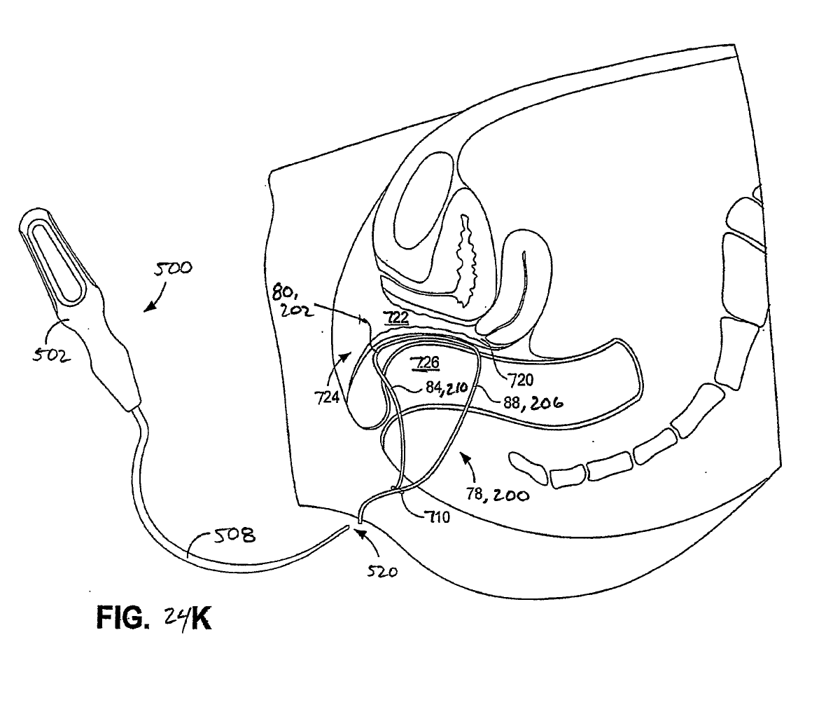

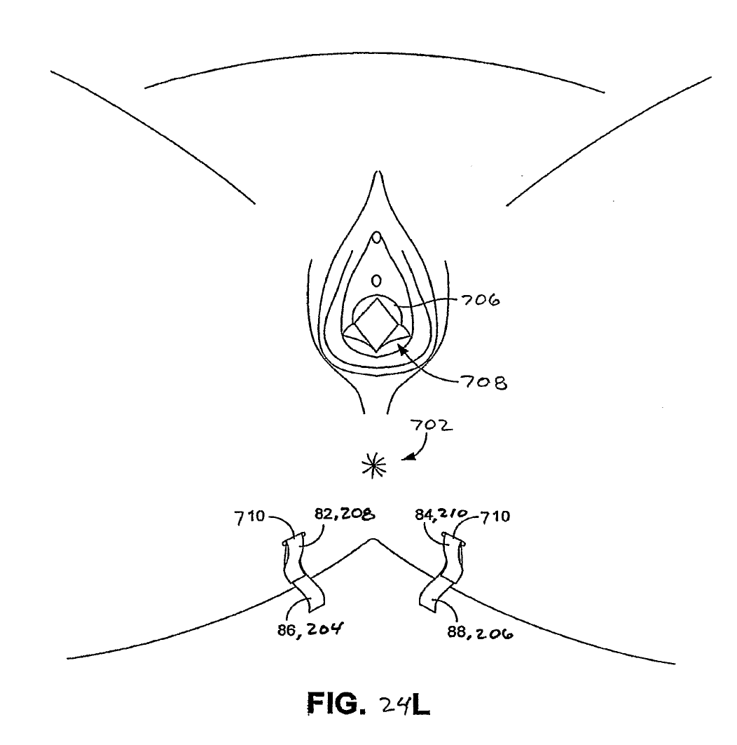

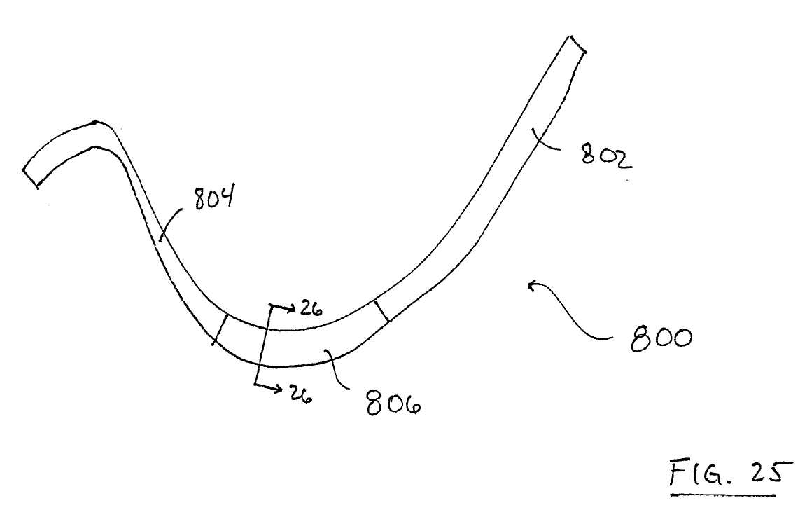

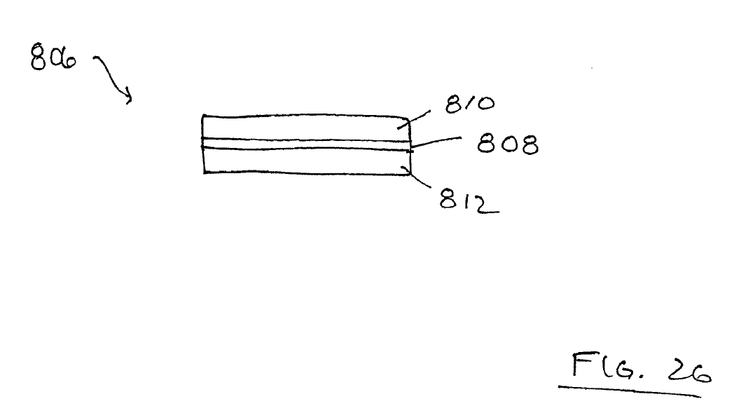

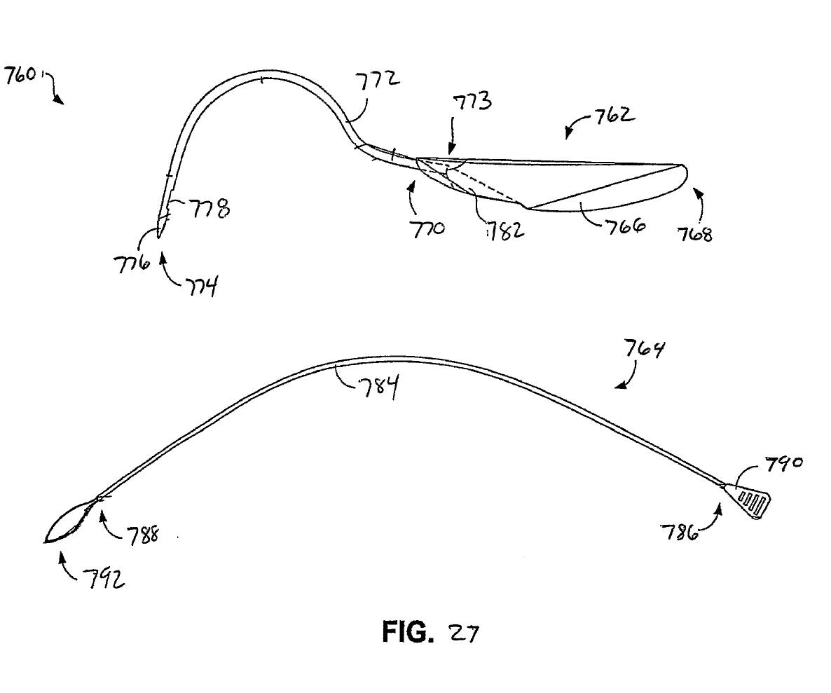

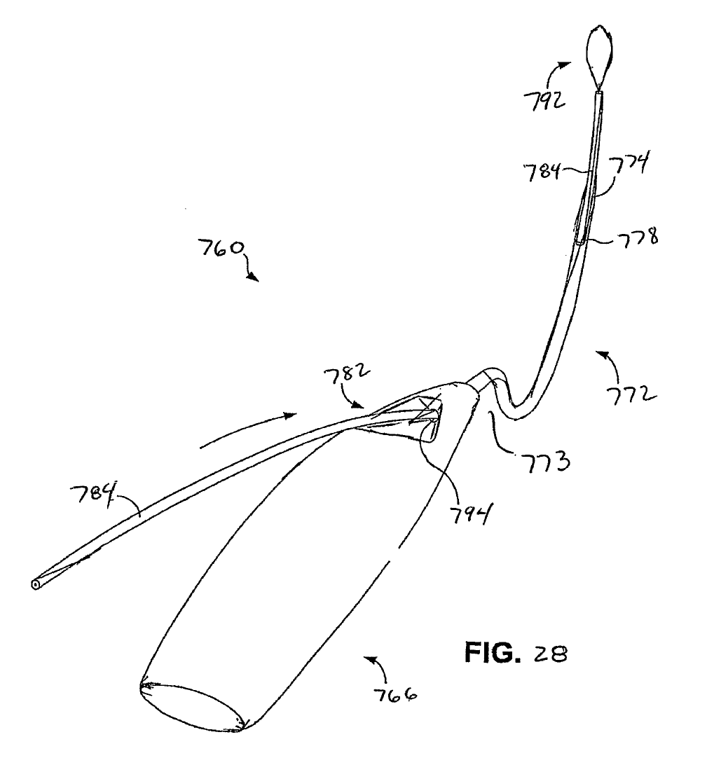

Although the above described implantation procedures reference the use of exemplary introducer needles, it should be appreciated that other suitable introducers and implantation methodologies may be employed, as the present invention is not limited in this respect. Accordingly, an alternative introducer system comprises a snare that can be extended from a tip of an introducer needle to a position outside of the body when the tip is positioned at a point within the body, such as within vagina. In such a case, an implant can be coupled to the extended snare and the snare can then be retracted to pull the implant through the body and at least to the tip of the introducer needle. In some embodiments, both the snare and the implant can further be drawn through the introducer needle such that the implant traverses the passage formed by the introducer needle without direct contact with the tissues of the passage, thereby reducing irritation to the soft tissues in which the passage is formed. One example of such a snare system is shown in FIGS. 27 and 28.

As indicated in FIGS. 27 and 28, the system 760 includes an introducer 762 and a snare 764. The introducer 762 comprises a handle 766 that includes a proximal end 768 and a distal end 770. A needle 772 extends from the distal end 770 of the handle 766. At least a portion of the needle 772 is curved. The needle includes a distal end 773 and a distal end 774. Formed at the distal end 774 is a blunt point or tip 776 that is configured to dissect soft tissue as the needle 772 is passed through the body. The needle 772 is hollow so as to form a cannula through which the snare 764 can be passed. More particularly, the needle 772 forms an inner lumen that extends from a first opening 778 of the needle to a second opening 780 of the needle. The first opening 778 is positioned adjacent the distal end 774 and the second opening 780 is positioned adjacent the proximal end 773. The second opening 780 is in open communication with a port 782 that is formed in the handle 766. As is described in greater detail below, the snare 764 can be passed through the port 782 and the second opening 780 to position the snare within the needle 772.

The snare 764 comprises an elongated shaft 784 having a proximal end 786 and a distal end 788. The shaft 784 is flexible so as to enable the shaft to easily adapt to the contours of the needle inner lumen and any body passages along which the snare is to travel. In some embodiments, the shaft 784 comprises a hollow tube through which a wire passes. In such cases, the shaft 784 can be constructed of a suitable flexible biocompatible material, such as a polymeric material. In other embodiments, the shaft 784 is solid and can be made of a polymeric material or a metal material, such as stainless steel or nitinol.

Provided at the proximal end 786 of the snare 764 is a grip element 790 that, as described below, is used to manipulate the snare relative to the introducer 762. Provided at the distal end 788 of the snare 764 is an implant coupling element 792 that is configured to couple to and secure an implant that is to be positioned with the body. In the illustrated embodiment, the coupling element 792 is formed as a loop. Such a loop can be formed from a flexible wire constructed of a polymeric or metal material, such as nitinol. In some embodiments, the shaft 784 and the coupling element 792 comprise a unitarily-formed element, such as an elongated wire that extends from the gripping element 790 and terminates in a loop. In such cases, the shaft 784 need not comprise a tube.

With the above-described system configuration, the snare 764 can be inserted through the port 782 and orifice 794 of the introducer handle 766, moved into the inner lumen of the introducer needle 772, pushed through the needle inner lumen, and made to exit the needle through the first opening 778. The result of that process is illustrated in FIG. 28.

Rather than have the surgeon manually move the snare through the introducer, the snare may be incorporated into the introducer 762 such that the snare is deployable and retractable upon actuation of a slide element, as the present invention is not limited in this respect.

Other suitable implantation tools may be employed, such as those described in PCT Application No. PCT/US2006/030581, filed Aug. 3, 2006; PCT Application No. PCT/US2006/030369, filed Aug. 3, 2006; PCT Application No. PCT/US2006/030370, filed Aug. 3,2006; and PCT Application No. PCT/US2006/062639, filed Dec. 28, 2006; the disclosures of which are all incorporated herein by reference in their entirety.

As mentioned above, the implant may be shaped for use in treating urinary incontinence. In one such embodiment, the implant is shaped as a strip of a material in the form of a sling. As shown in FIGS. 25 and 26, the sling 800 includes a central support section 806 adapted to face the urethra and is flanked by anchoring arms 802, 804. As best shown in FIG. 26, in one embodiment, the central section 806 includes a support layer 808 sandwiched between two layers of natural material 810, 812. It should be appreciated that only a single layer of natural material may be employed to face the urethra, as the present invention is not limited in this respect. Also, it should be appreciated that any of the above-noted features regarding the central section, the anchoring sections, the materials, the attachment arrangements, the fenestrations through the natural layer, and/or other features described above with regard to the anterior and posterior implants may be employed in or with the sling 800, as the present invention is not limited in this respect.

It should be understood that the foregoing description of the invention is intended merely to be illustrative thereof and that other embodiments, modifications, and equivalents of the invention are within the scope of the invention recited in the claims appended hereto. Further, the prosthesis described above includes various features that may be employed singularly or in any suitable combination.

What is claimed:

1-51. (canceled)

52. An implant comprising:
a body portion configured to support an organ, the body portion defining a central axis and a first end area at one end of the central axis and a second end area at an opposite end of the central axis, the body portion having lateral side edges;
a projection extending from the first end area of the body portion in a direction substantially parallel to the central axis, the projection having lateral side edges and an end opposite the first end area; and,
a first pair of arms extending from the body portion on opposite sides of the first end area and a second pair of arms extending from the body portion on opposite sides of the second end area, wherein each arm has a folded end portion.

53. The implant according to claim 52, further comprising a layer of natural material disposed on a first surface of the body portion and the projection.

54. The implant according to claim 53, wherein the folded end portion of an arm is folded toward a surface of the body portion comprising the natural layer of material.

55. The implant according to claim 52, wherein a crease defining the folded end portion of an arm of the first pair of arms is located approximately 50 mm from an end of the arm and wherein a crease defining the folded end portion of an arm of the second pair of arms is located approximately 40 mm from an end of the arm.

56. The implant according to claim 52, wherein the projection has a length of approximately 4 mm between the first end area and the end of the projection as measured along the central axis.

57. The implant according to claim 52, wherein the body portion and the projection comprise relatively soft mesh material and the arms comprise relatively coarse mesh material.

58. The implant according to claim 57, wherein a width of the relatively soft material is approximately 40 mm.

59. The implant according to claim 53, wherein the layer of natural material comprises a plurality of apertures formed therethrough.

60. The implant according to claim 59, wherein the layer of natural material is attached to the body portion and the projection with criss-crossing stitches resulting in a checkerboard pattern.

61. The implant according to claim 60, wherein a spacing between adjacent longitudinal stitch lines and adjacent lateral stitch lines is in a range between approximately 5 mm and approximately 10 mm.

62. The implant according to claim 60, wherein the stitch pierces through the natural layer of material at approximately every 3-4 mm.

63. The implant according to claim 52, wherein each arm of the first pair of arms extends substantially perpendicular to the central axis and wherein each arm of the second pair of arms extends at an angle relative to the central axis.

64. An implant comprising:
a support layer comprising:
a body portion configured to support an organ, the body portion defining a central axis and a first end area at one end of the central axis and a second end area at an opposite end of the central axis, the body portion having lateral side edges;
a projection extending from the first end area of the body portion in a direction substantially parallel to the central axis, the projection having lateral side edges and an end opposite the first end area; and
at least three anchoring arms extending from the body portion, with at least one first arm extending outward from the first end area and at least one second arm extending outward from the second end area; and
a layer of natural material disposed on a first surface of at least a portion of the support layer, wherein the first layer of natural material is spaced from the lateral side edges of both the body portion and the projection.

65. The implant according to claim 64, wherein the first layer of natural material is spaced a distance in a range between approximately 3 mm and approximately 7 mm from the lateral side edges of both the body portion and the projection.

66. The implant according to claim 64, wherein the layer of natural material extends over an entire length of the body portion and projection.

67. The implant according to claim 64, wherein the projection has a length of approximately 4 mm between the first end area and the end of the projection as measured along the central axis.

68. The implant according to claim 64, wherein the body portion comprises relatively soft mesh material and the arms comprise relatively coarse mesh material.

69. The implant according to claim 64, wherein each arm has a folded end portion.

70. The implant according to claim 69, wherein each arm has a folded end portion that is folded toward a surface of the body portion comprising the natural layer of material.

71. The implant according to claim 64, further comprising an indicator on the body portion configured to indicate a center line of the body portion, wherein the indicator is visible through the layer of natural material upon rehydration of the layer of natural material.

72-83. (canceled)
